(19) United States
(12) Patent Application Publication
(10) Pub. No.: US 2015/0339447 A1
(43) Pub. Date: Nov. 26, 2015

(54) MEDICAL ASSISTANCE DEVICE, OPERATION METHOD OF MEDICAL ASSISTANCE DEVICE, NON-TRANSITORY COMPUTER-READABLE RECORDING MEDIUM, AND MEDICAL ASSISTANCE SYSTEM

(71) Applicant: FUJIFILM CORPORATION, TOKYO (JP)

(72) Inventors: YUSUKE KITAGAWA, KANAGAWA (JP); SHOJI KANADA, KANAGAWA (JP)

(21) Appl. No.: 14/715,615

(22) Filed: May 19, 2015

(30) Foreign Application Priority Data
May 22, 2014 (JP) 2014-106172

(51) Int. Cl. G06F 19/00; A61B 5/00

(52) U.S. Cl. CPC G06F 19/345; A61B 5/7264; A61B 5/743

(57) ABSTRACT

There is provided a medical assistance device that can provide optimal diagnostic assistance information within a range of display items viewable by a doctor in medical information and can provide a development environment in which it is easy to efficiently improve a diagnostic assistance program to provide diagnostic assistance information. A medical assistance server includes an association setting unit and a parameter output unit. The association setting unit can set display items displayed on a display screen in medical information of a patient and a diagnostic assistance program for outputting diagnostic assistance information so as to be associated with each group unit of medical care. The parameter output unit outputs all pieces of first medical information corresponding to the display items, as parameters of the diagnostic assistance program, in a form in which all of the pieces of the first medical information are identifiable as the display items.

### CROSS-REFERENCE TO RELATED APPLICATIONS

The present application claims priority under 35 U.S.C. §119 to Japanese Patent Application No. 2014-106172, filed on May 22, 2014, all of which are hereby expressly incorporated by reference into the present application.

### BACKGROUND OF THE INVENTION

1. Field of the Invention

The present invention relates to a medical assistance device to assist a medical examination, an operation method of a medical assistance device, a non-transitory computer-readable recording medium, and a medical assistance system.

2. Description of the Related Art

In the medical field, various kinds of medical assistance systems for displaying medical information, such as examination data or measurement data acquired during the diagnosis of a patient, or for analyzing the medical information are known (refer to JP2003-271924A and JP2009-199598A). As one of the medical assistance systems, JP2003-271924A discloses an image processing system that displays or analyzes an examination image obtained by image examination, such as computed tomography (CT) examination or magnetic resonance imaging (MRI) examination. In the image processing system disclosed in JP2003-271924A, image processing programs are divided into, for example, a basic program for executing basic image processing commonly performed regardless of organs (for example, a lung or a liver), such as display of an image, and a dedicated application program (hereinafter, referred to as a dedicated application) for executing dedicated image processing for each organ, and it is possible to combine various kinds of dedicated applications with the basic program (paragraphs 0015, 0014, 0027, 0040, 0062, and 0103). The dedicated application is a program for outputting diagnostic assistance information, such as a lung cancer screening result, as a processing result by analyzing an examination image (medical information), for example.

According to such a configuration, the only thing to do in the medical facility after the introduction of the basic program is to add the dedicated application according to the purpose. Therefore, compared with a case of introducing an image processing program set each time according to the purpose, it is possible to reduce the cost of introducing image processing programs (paragraphs 0006 and 0116). In addition, for application development companies that develop dedicated applications, it is possible to reduce the cost of developing the dedicated applications since it is not necessary to develop the basic program (paragraphs 0097, 0098, and 0118).

JP2009-199598A discloses a system for assisting the selection of a dashboard (display screen that functions as a user interface), which is suitable for displaying the medical information of a patient that has been selected by the user, from a plurality of dashboards (paragraphs 0002 and 0011). The dashboard is configured such that it is possible to customize display items to be displayed on the dashboard in the medical information (paragraphs 0018 and 0074 to 0082).

### SUMMARY OF THE INVENTION

Incidentally, there are a variety of scales of medical facilities from large-scale medical facilities, such as regional core hospitals, to small-scale medical facilities, such as clinics, and introduced medical equipment, such as testing equipment and measurement equipment, differs depending on the medical facility. If the medical equipment is different, the number or types of items of medical information (examination data or measurement data) that can be obtained are also different. Therefore, display items of medical information that can be displayed on the display screen of a viewer terminal (viewing terminal) to display the medical information are also different. For example, in medical facilities that have introduced specific physiological testing equipment, it is possible to obtain the examination values of the specific physiological testing equipment. However, in medical facilities where the specific physiological testing equipment is not prepared, such examination values cannot be set on the display items since it is not possible to obtain the examination values. In addition, in medical facilities where only a general X-ray imaging apparatus is provided as image examination equipment, it is not possible to obtain a tomographic image based on CT examination as examination data. Therefore, in such medical facilities, it is not possible to set the tomographic image on the display items.

Even in the same medical facility, medical equipment differs depending on the department. In this case, since the number or types of items of medical information that can be obtained may be different, display items may also differ depending on the department. In addition, according to a medical facility or a department or even according to a disease in the same department, the basic policy on which item of medical information is to be seen, as a rule, in order to perform diagnosis may be different. In this case, display items may be different depending on the medical facility, department, disease, or the like. In recent years, with the increase of multiple diseases due to aging, the number of display items related to diseases also tends to increase, and display items that should be seen according to the condition of each patient may be different. Here, the concept including a medical entity, such as a medical facility or a department, and a medical examination target, such as a patient or a disease, is referred to as a group unit of medical care.

Thus, in the medical field, display items of medical information displayed on the viewer terminal may differ depending on the group unit of medical care, and the range of display items set so as to be viewable by a doctor also changes with the group unit of medical care. In addition, if the basic policy on the medical examination is different, the setting of the range of display items may change according to the policy even if information that can be obtained is the same since the medical equipment and the like are the same. Since the diagnosis is performed in the range of display items that can be viewed by a doctor, it is preferable that the diagnostic assistance information provided to the doctor by the medical assistance system can also be used in the range of display items. For example, in a case where the diagnostic assistance information prompts the monitoring of a change in a certain examination value, unless the examination value is included in display items set as a basic policy on the medical examination of the medical facility, it is difficult to refer to the diagnostic assistance information.

In addition, when advances in medical research or innovation in medical technology, such as development of new drugs or establishment of new knowledge, is made, diagnostic methods or treatment methods based on evidence (diagnostic methods or treatment methods proved to be good by research) also change daily.

Furthermore, with the development of information technology in recent years, also in the medical field, a large amount of various kinds of information have been collected. In other words, the accumulation of so-called big data is in progress. Through the use of such big data, diagnostic methods or treatment methods have also come to be present in large quantities for each department or disease, and medical individualization is also in progress. In order to respond to such diversification or evolution of diagnostic methods or treatment methods, it is necessary to quickly develop a large number of diagnostic assistance programs for providing a diagnostic assistance function that can be applied to medical assistance systems. In order to quickly develop a large number of diagnostic assistance programs, it is preferable to encourage the participation of a number of unspecified development companies so that efficient development is performed in society as a whole.

In addition, since display items in the viewer terminal that displays medical information are customized and set according to the group unit of medical care within certain constraints as described above, it is preferable to provide optimal diagnostic assistance information within the constraints. However, development companies that develop diagnostic assistance programs to provide diagnostic assistance information could not know display items set for each group unit of medical care (for example, a medical facility, a department, a disease, or a patient) in the viewer terminal in the medical field until now. In order to make it easier to use each diagnostic assistance program in the medical field, it is preferable to develop the diagnostic assistance program for offering optimal diagnostic assistance with the top priority being given to the range of display items of the viewer terminal.

When diagnosis or treatment content is changed, display items set so as to be viewable by the doctor are also changed in a medical facility or a department since the basic policy of the medical facility or medical examination is reformed or modified. In order to provide diagnostic assistance information involving the latest diagnostic methods or the latest treatment methods or to respond to display item changes, it is necessary to continuously improve the diagnostic assistance program. In order to perform continuous improvement of the diagnostic assistance program efficiently, it is preferable that the development company of the diagnostic assistance program has a system in which display item changes can be efficiently checked.

In the systems disclosed in JP2003-271924A and JP2009-199598A, there is neither description nor suggestion for the above issues or their solutions.

It is an object of the invention to provide a medical assistance device that can provide optimal diagnostic assistance information within a range of display items viewable by a doctor in medical information and can provide a development environment in which it is easy to efficiently perform development or improvement of a diagnostic assistance program to provide diagnostic assistance information, an operation method of a medical assistance device, a non-transitory computer-readable recording medium, and a medical assistance system.

In order to solve the aforementioned problem, a medical assistance device of the invention includes an association setting unit, a medical information acquisition unit, and a parameter output unit. The association setting unit can set display items displayed on a display screen including medical information of a patient and a diagnostic assistance program for outputting diagnostic assistance information to assist diagnosis of the patient so as to be associated with each other for each group unit of medical care. The medical information acquisition unit acquires the medical information. The parameter output unit outputs all pieces of first medical information corresponding to at least the display items in the medical information acquired by the medical information acquisition unit, as parameters to be input to the diagnostic assistance program, in a form in which all of the pieces of the first medical information are identifiable as the display items. Here, the outputting in the identifiable form means outputting only the first medical information or outputting the first medical information with identification information being given thereto. As the identification information, it is possible to use any form, such as an identification flag and an item name.

The group unit of medical care includes at least one of a medical facility, a department in a medical facility, a disease, a patient, and a medical practice phase indicating a progress phase of medical treatment or a disease. Preferably, the first medical information and the diagnostic assistance information are displayed on the display screen.

Preferably, the parameter output unit outputs the first medical information and second medical information, which does not correspond to the display items in the medical information, as parameters of the diagnostic assistance program.

Preferably, the diagnostic assistance program has a function of outputting content regarding the first medical information and content regarding the second medical information as the diagnostic assistance information, and the diagnostic assistance program has a function of outputting at least one of a message, which prompts a user to set at least one item of the second medical information to the display items, and evidence information as a basis of the message in case where the content regarding the second medical information is included in the diagnostic assistance information.

The medical information may include a plurality of types of examination images captured by a plurality of modalities, and the parameter output unit may output the plurality of types of examination images as the first medical information or the second medical information.

In case where there is image analysis information obtained by analyzing the examination images, the parameter output unit may output the image analysis information together with the plurality of types of examination images.

It is preferable to further include a program control unit that controls starting of the diagnostic assistance program and the parameter output of the parameter output unit for the diagnostic assistance program to be started.

It is preferable to further include a designated information receiving unit that receives group unit of medical care designation information for designating the group unit of medical care, the group unit of medical care designation information being input through the display screen. Preferably, the program control unit determines the diagnostic assistance program associated with the designated group unit of medical care based on the group unit of medical care designation information, and controls starting of the determined diagnostic assistance program and output of the parameters for the diagnostic assistance program to be started.

Preferably, in case where the group unit of medical care is a disease, the designated information receiving unit receives designation of multiple diseases. Preferably, in case where the multiple diseases are designated, the program control unit preferentially starts a diagnostic assistance program suitable for the multiple diseases among a plurality of the diagnostic assistance programs.

Preferably, the program control unit starts the diagnostic assistance program, and executes a batch process for collectively outputting the diagnostic assistance information regarding a plurality of patients.

Preferably, the diagnostic assistance program includes a diagnostic program having a function of outputting the diagnostic assistance information regarding one patient of interest, who is to be diagnosed, and reference information regarding other patients.

Preferably, the program control unit periodically starts a monitoring program having a function of monitoring whether or not the first medical information is in a normal range, and performs a warning process in case where it is determined that the first medical information is not in the normal range.

It is preferable to further include an anonymization unit that anonymizes personal information of a patient in the first medical information.

It is preferable to further include a screen data generation unit that generates the display screen for displaying the first medical information based on the medical information acquired by the medical information acquisition unit; and an output control unit that outputs the screen data.

Preferably, a diagnostic assistance information display region for displaying the diagnostic assistance information is provided on the display screen. Preferably, a start display showing that the diagnostic assistance program is starting is performed on the display screen. Preferably, when a plurality of the diagnostic assistance programs are starting, a start display showing that the plurality of diagnostic assistance programs are starting is performed.

Preferably, a message asking whether or not to allow starting of the diagnostic assistance program is displayed on the display screen before starting the diagnostic assistance program.

An operation method of a medical assistance device of the invention includes an association setting step, a medical information acquisition step, and a parameter output step. In the association setting step, it is possible to set display items displayed on a display screen including medical information of a patient and a diagnostic assistance program for outputting diagnostic assistance information to assist diagnosis of the patient so as to be associated with each other for each group unit of medical care. In the medical information acquisition step, the medical information is acquired. In the parameter output step, all pieces of first medical information corresponding to at least the display items in the medical information acquired by the medical information acquisition unit are output, as parameters of the diagnostic assistance program, in a form in which all of the pieces of the first medical information are identifiable as the display items.

A medical assistance program of the invention is a medical assistance program causing a computer to function as a medical assistance device, and includes an association setting step, a medical information acquisition step, and a parameter output step. In the association setting step, it is possible to set display items displayed on a display screen including medical information of a patient and a diagnostic assistance program for outputting diagnostic assistance information to assist diagnosis of the patient so as to be associated with each other for each group unit of medical care. In the medical information acquisition step, the medical information is acquired. In the parameter output step, all pieces of first medical information corresponding to at least the display items in the medical information acquired by the medical information acquisition unit are output, as parameters of the diagnostic assistance program, in a form in which all of the pieces of the first medical information are identifiable as the display items.

A medical assistance system of the invention includes a diagnostic assistance program and a medical assistance device that executes the diagnostic assistance program. The diagnostic assistance program outputs diagnostic assistance information to assist diagnosis of a patient. The medical assistance device includes an association setting unit, a medical information acquisition unit, and a parameter output unit. The association setting unit can set display items displayed on a display screen including medical information of a patient and the diagnostic assistance program so as to be associated with each other for each group unit of medical care. The medical information acquisition unit acquires the medical information. The parameter output unit outputs all pieces of first medical information corresponding to at least the display items in the medical information acquired by the medical information acquisition unit, as parameters to be input to the diagnostic assistance program, in a form in which all of the pieces of the first medical information are identifiable as the display items.

According to the invention, it is possible to provide a medical assistance device that can provide optimal diagnostic assistance information within a range of display items viewable by a doctor in medical information and can provide a development environment in which it is easy to efficiently perform development or improvement of a diagnostic assistance program to provide diagnostic assistance information, an operation method of a medical assistance device, a medical assistance program, and a medical assistance system.

### BRIEF DESCRIPTION OF THE DRAWINGS

FIG. 1 is an explanatory diagram showing a medical information system.

FIG. 2 is an explanatory diagram showing medical information recorded in the electronic medical record.

FIG. 3 is an explanatory diagram showing medical information recorded in an image server.

FIG. 4 is an explanatory diagram showing the flow of main information of the medical information system.

FIG. 5 is a block diagram showing the electrical configuration of a computer used as a medical assistance server or the like.

FIG. 6 is an explanatory diagram showing the functional overview of a client terminal.

FIG. 7 is an explanatory diagram showing an example of a display screen.

FIG. 8 is an explanatory diagram of a display item setting screen.

FIG. 9 is an explanatory diagram of an assistance program setting screen.

FIG. 10 is an explanatory diagram of a display item setting table.

FIG. 11 is an explanatory diagram showing the functional overview of a medical assistance server.

FIG. 12 is an explanatory diagram showing the functional overview of a program control unit.

FIG. 13 is a flowchart showing the procedure of a setting process.

FIG. 14 is a flowchart showing the distribution procedure of medical information and diagnostic assistance information.

FIG. 15 is an explanatory diagram of first medical information and second medical information.

FIG. 16 is an explanatory diagram of an embodiment of outputting the second medical information in addition to the first medical information.

FIG. 17 is an explanatory diagram of information related to the second medical information provided as diagnostic assistance information.

FIG. 18 is an explanatory diagram of a display item setting table when the group unit of medical care is a department.

FIG. 19 is an explanatory diagram of a display item setting table when the group unit of medical care is a patient.

FIG. 20 is an explanatory diagram of a display item setting table when the group unit of medical care is a medical practice phase.

FIG. 21 is an explanatory diagram of a display item setting table when the group unit of medical care is a combination of a department and a medical practice phase.

FIG. 22 is a flowchart showing the procedure of a batch process for diagnostic assistance information calculation.

FIG. 23 is a flowchart showing the procedure of creating reference information in addition to medical information.

FIG. 24 is a flowchart showing the procedure of a monitoring process.

FIG. 25 is an explanatory diagram of an anonymization unit.

FIG. 26 is an explanatory diagram of a medical assistance server when a screen distribution function is separated therefrom.

FIG. 27 is an explanatory diagram when a client terminal functions as a medical assistance device.

FIG. 28 is an explanatory diagram of an example in which a medical assistance server is shared by a plurality of medical facilities.

FIG. 29 is an explanatory diagram of an example in which a medical assistance server and a distribution server are separately provided so as to be shared by a plurality of medical facilities.

FIG. 30 is an explanatory diagram of an example when a medical assistance server is installed outside a medical facility and a distribution server is installed in a medical facility.

FIG. 31 is an explanatory diagram of a display item setting table when the group unit of medical care is a medical facility.

FIG. 32 is an explanatory diagram of an inquiry message asking whether or not to allow the starting of a diagnostic assistance program.

FIG. 33 is an explanatory diagram of a starting display of a diagnostic assistance program.

### DESCRIPTION OF THE PREFERRED EMBODIMENTS

### First Embodiment

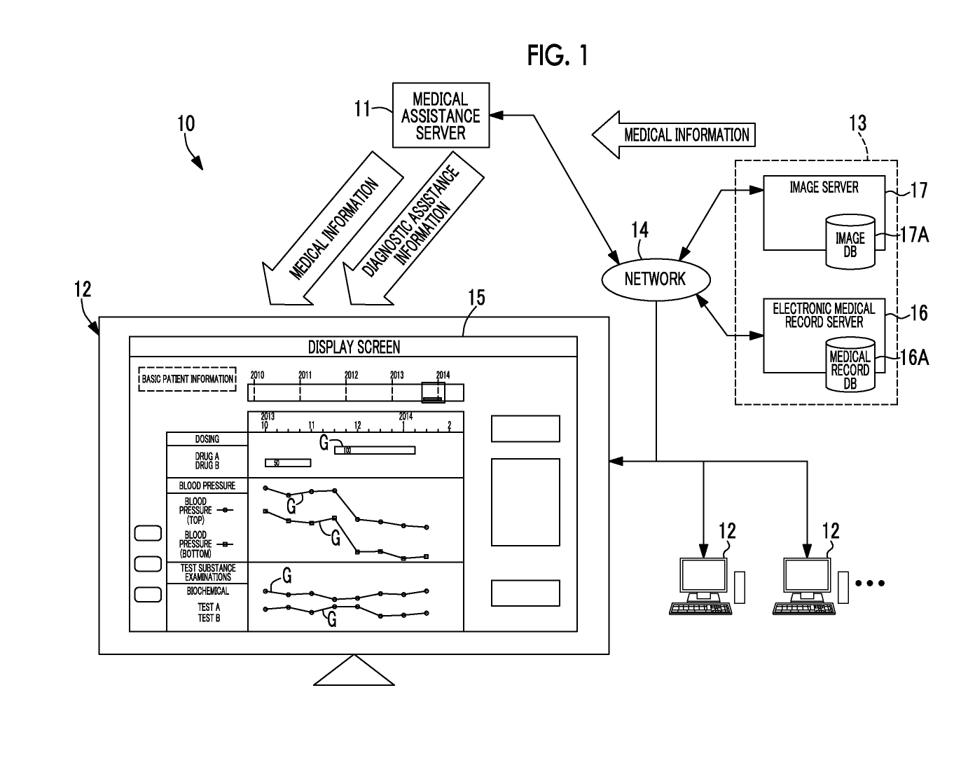

A medical information system 10 shown in FIG. 1 is a computer system used for the management and use of medical information in medical facilities, such as a hospital. The medical information system 10 is configured to include a medical assistance server 11, a client terminal 12, a server group 13, and a network 14 that communicably connects these to each other. The server group 13 is a server that stores the medical information of a patient, and an electronic medical record server 16, an image server 17, and the like are included in the server group 13. The network 14 is a local area network (LAN) provided in the hospital, for example.

The client terminal 12 is a terminal installed in each department, such as internal medicine, surgery, otolaryngology, and ophthalmology, and is operated by the doctor of each department. The client terminal 12 has a function of accessing the electronic medical record server 16 to input or view electronic medical records. As the electronic medical records, medical information including medical examination records such as interview and diagnostic content, examination values of medical examinations or measurement values of vital signs, and treatment records such as treatment and surgery, is input. The client terminal 12 has a function of accessing the image server 17 to view examination images, such as X-ray images. Thus, the client terminal 12 functions as a viewer terminal for viewing the medical information.

In addition, the client terminal 12 has a function of accessing the medical assistance server 11 to receive the distribution of the medical information of a patient and display the distributed medical information on a display screen 15 and view it. Unlike a medical record display screen only for electronic medical records or an image display screen only for examination images, the display screen 15 can collectively display the examination values or measurement values of medical examinations and examination images, which are included in electronic medical records, in one screen. In the display screen 15, when there is a plurality of examination values or a plurality of measurement values, time-series data indicating a temporal change thereof is displayed in the form of, for example, a graph G.

The medical assistance server 11 acquires the medical information of a designated patient from the electronic medical record server 16 or the image server 17 in response to a distribution request including the designation of a patient from the client terminal 12, and distributes the acquired medical information to the requesting client terminal 12. In addition to the medical information of the patient, in order to assist the diagnosis by a doctor, the medical assistance server 11 can provide diagnostic assistance information available as a reference during the diagnosis.

The electronic medical record server 16 includes an electronic medical record database (hereinafter, referred to as a medical record database (DB)) 16A in which electronic medical records are stored. The image server 17 has an image DB 17A in which a plurality of examination images are stored, and is a so-called picture archiving and communication system (PACS) server. The medical record DB 16A or the image DB 17A is a database that can be searched using a keyword, such as patient identification data (ID).

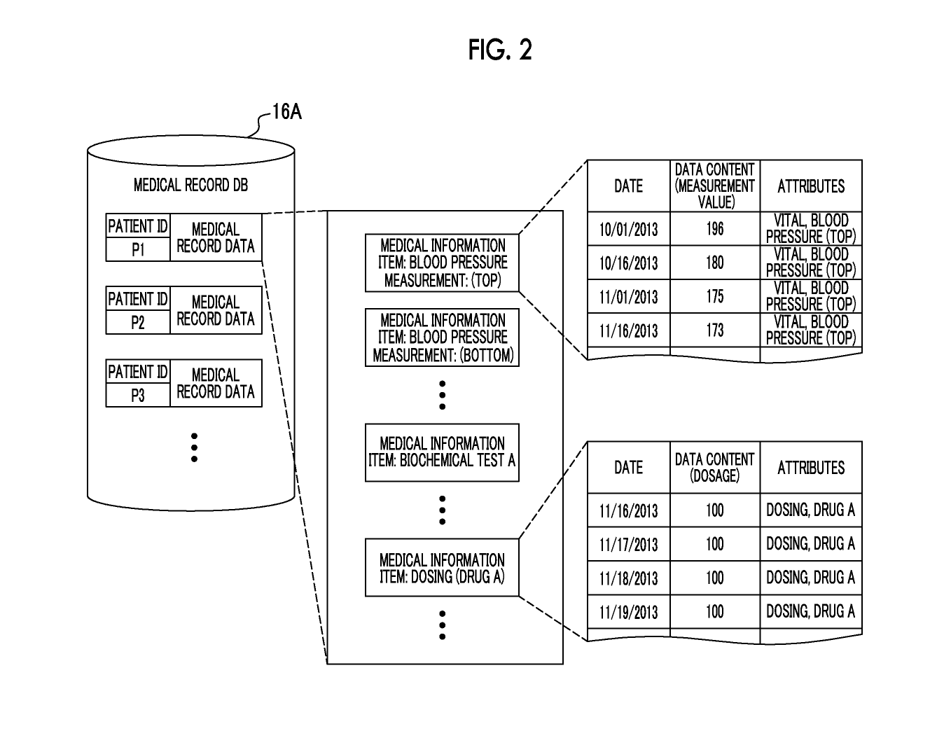

As shown in FIG. 2, medical record data in which the medical information of a patient is recorded is stored in the medical record DB 16A. Patient IDs (P1, P2, . . . ) are given to the medical record data, and the medical record data is managed in units of patients. The medical record data includes basic patient information, such as patient's name, date of birth, and sex and a patient ID, and medical information of patients.

Measurement values of vital signs, such as patient's heart rate, pulse, blood pressure, and body temperature, and examination values of clinical tests performed on the patient are included in the medical information. The clinical tests include test substance examinations, such as blood tests, biochemical tests, and physiological tests, such as electroencephalography. In addition, the content of a medical examination performed for a patient, specifically, the content of treatment, such as medication or surgery, and the content of the interview are included in the medical information. Thus, there is a plurality of items in the medical information. In FIG. 2, items, such as measurement values of blood pressure (top) and blood pressure (bottom), examination values of a biochemical test A, and dosage of drug A, are illustrated as medical information.

Information regarding the date and time such as an examination date or a measurement date, acquired data content (examination value or measurement value), and attributes are included in the record for one case of each item of medical information. The information of date and time is measurement date and time in the case of a measurement value, and is examination date and time in the case of an examination value, and is dosing date and time or prescription date and time in the case of a dosage. For dosing, there is a case where it takes time until the dosing effect appears. Accordingly, there is a case where dosing (taking drugs) over a predetermined period of time is indicated by one prescription, for example, such as “take a predetermined amount per day continuously for 5 days”. In this case, as shown in FIG. 2, date and time for which dosing is scheduled is recorded as date and time of dosing.

Attributes are information given to classify data, and is information indicating the attribute of each item of medical information. The attributes can also be used as a keyword for searching for the medical information. As attributes, for example, an item name (“blood pressure (top)”, “dosing”, and the like) of medical information and a category (“vital”, “dosing”) to which an item belongs are included. To the item of dosing, for example, a drug name “drug A” is given in addition to the item name of “dosing”.

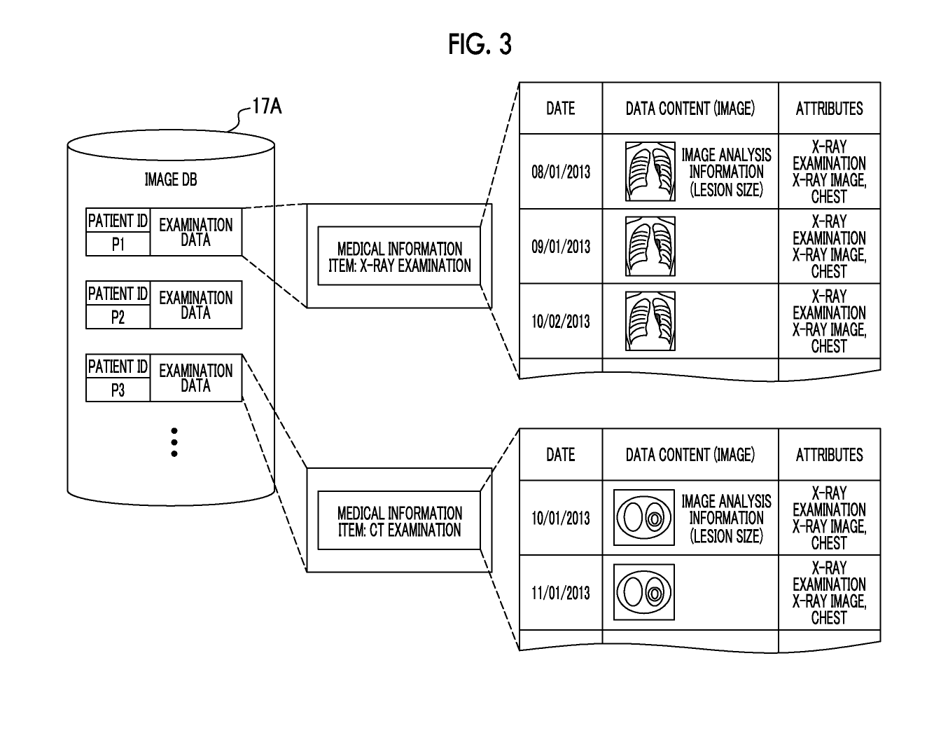

As shown in FIG. 3, examination data including a plurality of examination images captured by image examination, such as X-ray examination or CT examination, is stored in the image DB 17A. A patient ID is given to each examination image, so that the examination image can be searched based on the patient ID. In the image examination, as a plurality of tomographic images obtained by CT examination, a plurality of examination images may be captured in a single examination. The same examination ID is given to the plurality of examination images captured in a single examination, so that the plurality of examination images captured in a single examination is managed as examination images of one case. In addition, the examination images are also managed for each date and time in which image examination has been performed.

In the data content of the examination image, image analysis information obtained by analyzing the examination image may be included in addition to the data of the examination image. As the image analysis information, for example, there is information regarding the size of a lesion or the type of a lesion in the examination image. The image analysis information may be information calculated by image processing, or may be information based on the input of the content that a doctor determines through image observation. As attributes of the examination image, for example, information such as “X-ray examination” or “CT examination” indicating the examination type, “X-ray image” or “CT image” indicating the image type, and “chest” or “head” indicating the imaging part is given.

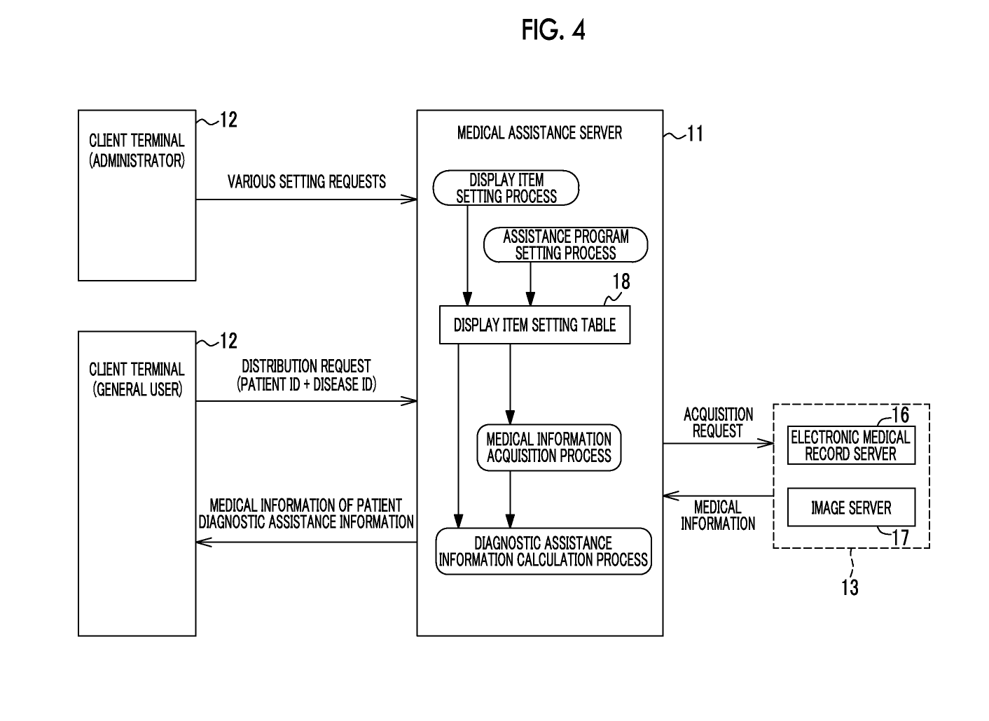

As shown in FIG. 4, the medical assistance server 11 has a setting function for performing a process of setting the display item (display item setting process) of the display screen 15 distributed to the client terminal 12 and a process of setting a diagnostic assistance program (assistance program setting process). The display item setting process is a process of setting display items displayed on the display screen 15 among all items of the medical information stored in the server group 13. The display item is set for each group unit of medical care. Here, the group unit of medical care is a concept including one of the medical entity such as a medical facility or a department, a medical examination target such as a patient or a disease, and the medical practice phase that is a progress phase of medical treatment or disease. In addition, the concept of a combination of these is also included in the group unit of medical care. For example, it is possible to set the combination of a patient and a disease as a group unit of medical care, such as setting a certain disease of a certain patient as one group unit of medical care.

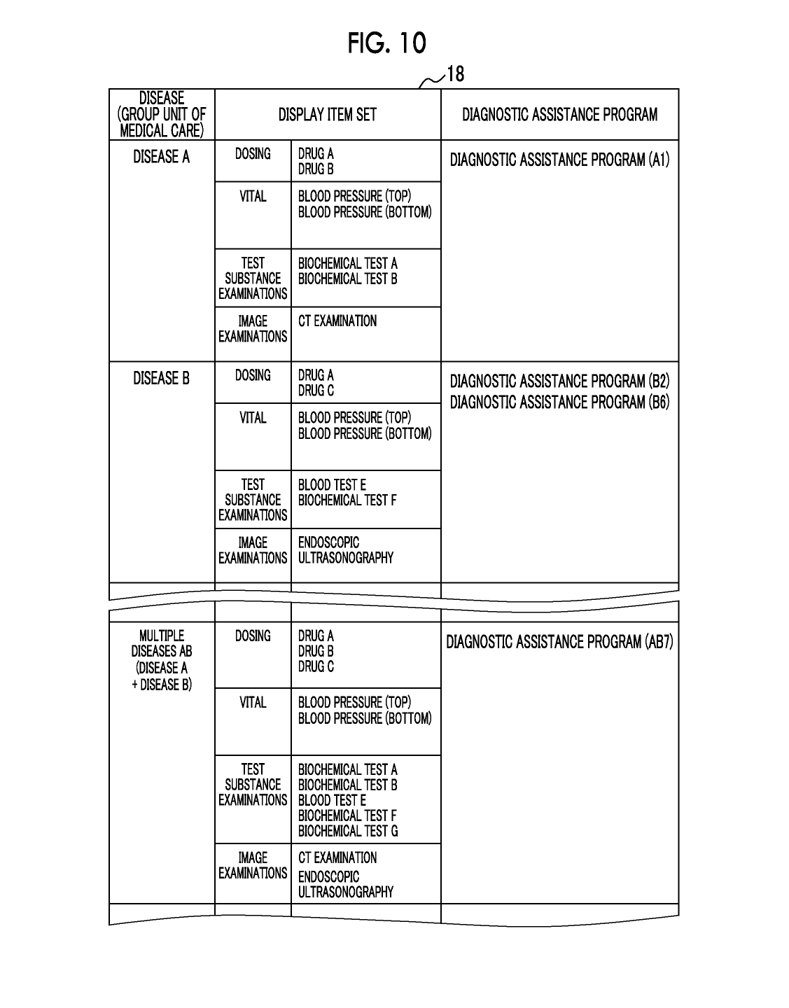

Items to be referred to during the diagnosis in the medical information may differ depending on the medical facility, department, patient, disease, medical practice phase, and the like. Therefore, in the medical assistance server 11, the items of medical information displayed on the display screen 15 can be set as display items for each group unit of medical care. The settings are recorded in a display item setting table 18 (refer to FIG. 10). In this example, the group unit of medical care is a disease. Accordingly, in the display item setting table 18, a display item is set for each disease.

Since a display item is determined based on the basic policy of a medical facility or department in many cases, display item setting authority is given to a doctor having management authority of the medical facility, for example. As shown in the diagrams, when a display item setting request from the administrator having management authority is transmitted from the client terminal 12, the medical assistance server 11 receives the setting request. Then, by performing the display item setting process according to the content of the setting request, the display item setting table 18 is updated. Since the setting authority is not given to a doctor (general user) other than the administrator, only the viewing of the set display item (only the viewing of the settings of the display item setting table 18) is allowed.

When displaying medical information, a doctor that is a general user designates a patient ID and a disease ID (a group unit of medical care in this example) in the client terminal 12, and instructs the medical assistance server 11 to transmit the medical information. In response to this instruction, the client terminal 12 transmits a distribution request including the designated patient ID and disease ID to the medical assistance server 11.

The medical assistance server 11 reads a display item corresponding to the disease ID with reference to the display item setting table 18. Then, the medical assistance server 11 transmits a request to acquire medical information corresponding to the display item corresponding to the disease ID, among all items of the medical information of the designated patient ID, to the server group 13, thereby performing medical information acquisition processing for acquiring the medical information. The medical assistance server 11 distributes the acquired medical information to the client terminal 12 that is a distribution requester.

Thus, by managing the display items for each disease (each group unit of medical care) using the display item setting table 18, it is possible to prevent an oversight of a basic display item that a doctor should see during the diagnosis for each disease.

In this example, the display item setting authority is not given to a doctor that is a general user. However, the doctor as a general user cannot just change the display item setting table 18, but can perform screen editing, such as temporarily adding a display item, on the display screen 15. Therefore, the doctor as a general user can also view an item (non-setting item) other than display items that are set so as to be associated with each group unit of medical care in the display item setting table 18. That is, although the display items are displayed on the display screen 15 in an initial state in which no screen editing operation has been performed, the non-setting item is not displayed on the display screen 15 in the initial state but can be temporarily displayed by performing a screen editing operation. Thus, the display of the non-setting item may be prohibited, or the display priority of the non-setting item may be set to be lower than that of the display item. In other words, it is preferable to set the display priority of the display item to be higher than that of the non-setting item.

As another method of setting the priority of the display item to be higher than that of the non-setting item, there is a method of changing the priority according to the arrangement order on the display screen 15, for example. In general, the display screen 15 is created so as to be able to be scrolled. In the case of a scrollable form, the priority of the display item is set to be higher than that of the non-setting item in connection with the arrangement order of the display screen 15. Therefore, for example, display items having a high arrangement order are displayed on the initial display screen 15 on which no scroll operation is required, and non-setting items having a low arrangement order can be displayed by the scroll operation.

Depending on the number of display items, all of the display items may not be shown on the initial display screen 15. In this case, a scroll operation is required to see the rest of the display items that are not shown on the initial display screen 15. In such a case, distinction between display items and non-setting items (where the display items end and where the non-setting items start) is not clear. Accordingly, it is preferable to clearly distinguish the display items and the non-setting items from each other by using a method of inserting a borderline in a boundary between a display item and a non-setting item arranged subsequent to the display item on the display screen 15, for example. Thus, by increasing the priority of the display items according to the arrangement order on the display screen 15 that can be scrolled, it becomes relatively easy to see the display items. As a result, it is possible to provide user friendliness for a doctor. In essence, display items according to the group unit of medical care are necessarily displayed on the display screen 15, and non-setting items other than the display items may be displayed or may not be displayed since the display of non-setting items does not have a significant effect on convenience.

The diagnostic assistance program is a program product for outputting the diagnostic assistance information by performing a calculation based on the medical information of a patient, and a plurality of types of diagnostic assistance programs are prepared according to the function. The assistance program setting process is a process of setting which diagnostic assistance program among a plurality of diagnostic assistance programs is to be used. The diagnostic assistance program is set so as to be associated with the display items which is set for each disease (each group unit of medical care).

Similar to the display item setting process, the assistance program setting process is also determined based on the basic policy of a medical facility or department in many cases. Accordingly, diagnostic assistance program setting authority is given to a doctor having management authority. The medical assistance server 11 receives a diagnostic assistance program setting request from the administrator having management authority, and performs the assistance program setting process. The assistance program setting information is recorded in the display item setting table 18. The diagnostic assistance program may be set for all of group unit of medical cares, or the diagnostic assistance program may be set only for some of the group unit of medical cares. As an example in which the diagnostic assistance program is set only for some of the group unit of medical cares, it is possible to consider an example in which the diagnostic assistance program is set only for a specific group unit of medical care. For example, the diagnostic assistance program is set only for a disease (group unit of medical care) whose diagnosis is difficult in particular.

After receiving the medical information distribution request from the client terminal 12, the medical assistance server 11 reads a disease ID designated from the distribution request. Then, the medical assistance server 11 selects a diagnostic assistance program, which is set so as to be associated with the disease ID, with reference to the display item setting table 18. The medical assistance server 11 executes the selected diagnostic assistance program, and performs a diagnostic assistance information calculation process for calculating the diagnostic assistance information. In the diagnostic assistance calculation process, the medical assistance server 11 executes the diagnostic assistance program by using the medical information of the patient, which corresponds to the display items set for each disease, as parameters. The calculated diagnostic assistance information is distributed to the client terminal 12 that is a distribution requester.

The medical assistance server 11, the client terminal 12, the electronic medical record server 16, and the image server 17 are configured by installing a control program, such as an operating system, or an application program, such as a client program or a server program, into a computer as a base, such as a personal computer, a server computer, or a workstation.

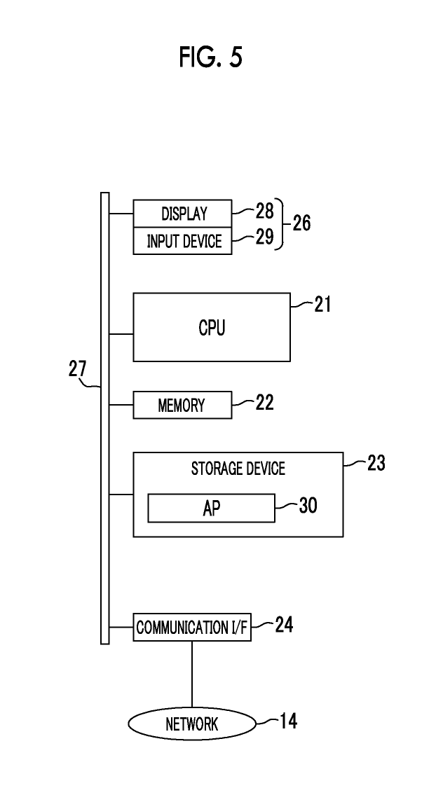

As shown in FIG. 5, computers that form the servers 11, 16, and 17 and the client terminal 12 have basically the same configuration, and each computer includes a central processing unit (CPU) 21, a memory 22, a storage device 23, a communication I/F 24, and an input/output unit 26. These are connected to each other through a data bus 27. The input/output unit 26 is configured to include a display (display unit) 28 and an input device 29, such as a keyboard or a mouse.

The storage device 23 is, for example, a hard disk drive (HDD), and a control program or an application program (hereinafter, referred to as an AP) 30 is stored in the storage device 23. In a server in which a DB is built, apart from the HDD to store a program, for example, a disk array formed by connecting a plurality of HDDs to each other is provided as the storage device 23 for DB. The disk array may be built in the main body of a server, or may be provided separately from the main body of a server and be connected to the main body of the server through a cable or a network.

The memory 22 is a work memory required when the CPU 21 executes processing, and is a random access memory (RAM). The CPU 21 performs overall control of each unit of the computer by loading the control program stored in the storage device 23 to the memory 22 and executing the processing according to the program. The communication I/F 24 is a network interface for transmission control through the network 14.

A client program, such as electronic medical record software for performing the viewing or editing of electronic medical records or viewer software for performing the viewing of an examination image or the display screen 15, is installed in the client terminal 12 as the AP 30. The viewer software may be dedicated software, or may be a general-purpose WEB browser, for example.

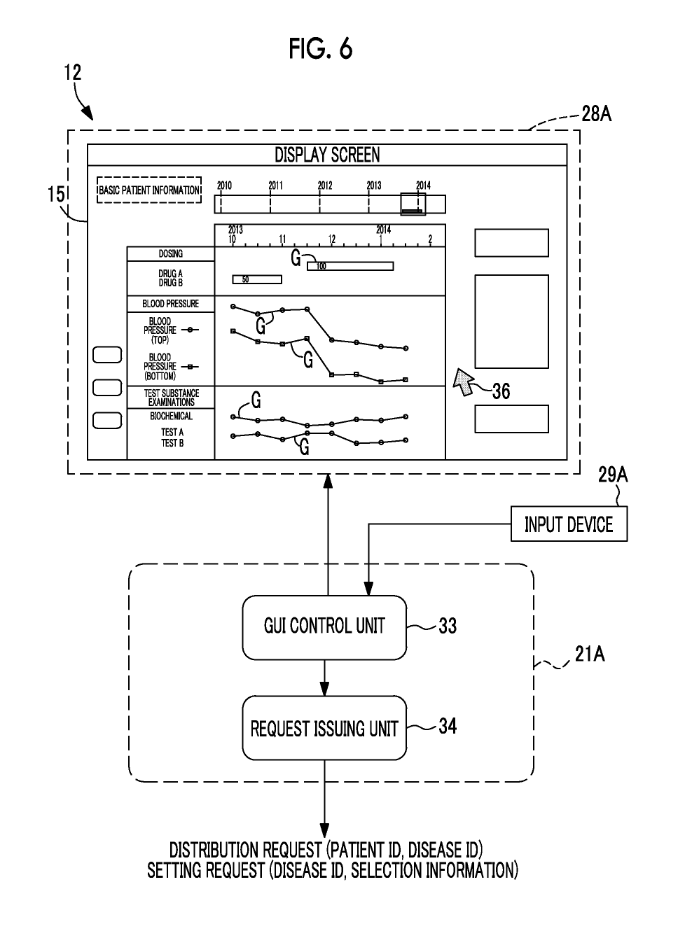

As shown in FIG. 6, when the viewer software for displaying the display screen 15 is activated in the client terminal 12, a startup screen with an operation function through a graphical user interface (GUI) is displayed on a display 28A of the client terminal 12, and a CPU 21A of the client terminal 12 cooperates with the memory 22 and the like to function as a GUI control unit 33 and a request issuing unit 34 that issues various requests for the medical assistance server 11. On the startup screen, operation instructions, such as the designation of a patient ID or a disease ID and a medical information distribution instruction, are given.

The medical assistance server 11 distributes screen data that forms the display screen 15. The screen data is formed by data written in a markup language, such as Extensible Markup Language (XML), for example. The GUI control unit 33 reproduces the display screen 15 based on the received screen data, and displays the reproduced display screen 15 on the display 28A. The display screen 15 also functions as an operation screen. The GUI control unit 33 performs screen control according to an operation instruction input from an input device 29A through the display screen 15, such as an operation button clicking operation using a pointer 36 of a mouse. The request issuing unit 34 receives the designation of a patient ID and a disease ID and a medical information distribution instruction through the GUI control unit 33, and issues a medical information distribution instruction.

In addition, an instruction to set display items or a diagnostic assistance program is also input through the display screen 15. The instruction to set display items or a diagnostic assistance program includes information for selecting the disease ID, display items, or diagnostic assistance program. After receiving the setting instruction through the GUI control unit 33, the request issuing unit 34 issues a setting request. The issued distribution request or setting request is transmitted to the medical assistance server 11 through the network 14.

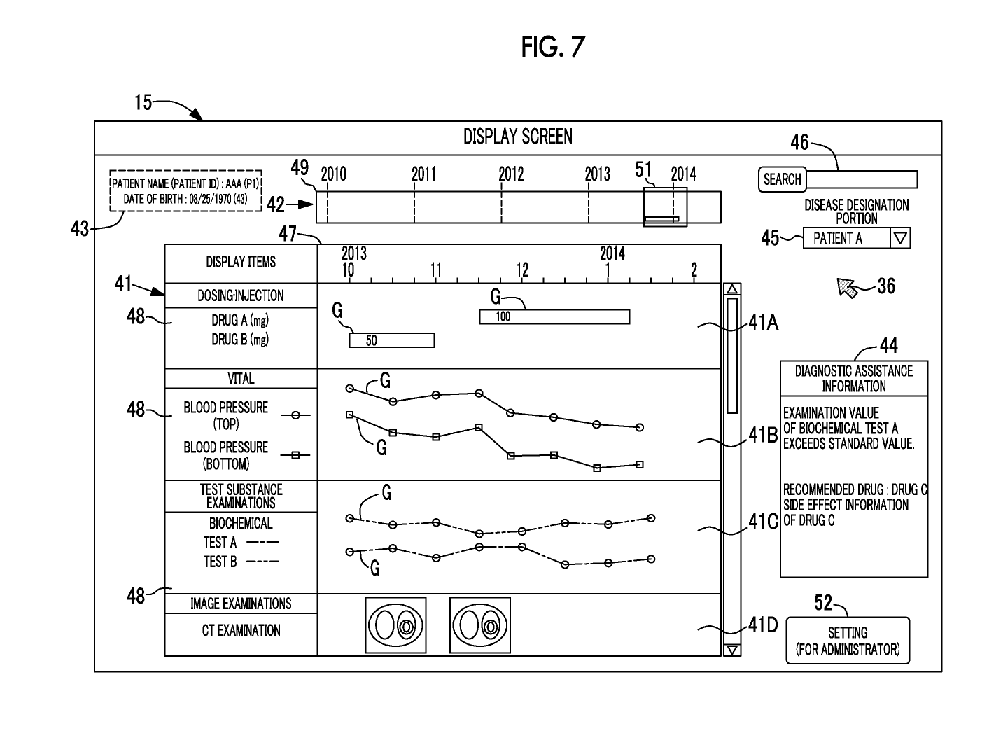

As shown in FIG. 7, the display screen 15 includes a first display region 41, a second display region 42, a basic patient information display region 43, a diagnostic assistance information display region 44, and a disease designation portion 45. Basic patient information, such as a patient name, a patient ID, and age, is displayed in the basic patient information display region 43. Designation of a patient ID is performed by entering the patient name or the patient ID to the basic patient information display region 43, for example.

The disease designation portion 45 is an operation portion for designating the type of disease. The disease designation portion 45 is configured such that a disease list is displayed, for example, in a pull-down menu format, and a disease is designated from the disease list by the operation of the pointer 36. A disease search portion 46 is provided above the disease designation portion 45. When the symptoms or the like of a patient are input to the disease search portion 46 as a search keyword, candidates of the disease predicted from the symptoms are searched for, and the search result is output. The output search result can be used to designate the disease.

The first display region 41 is a region where medical information is displayed. In the first display region 41, the horizontal axis indicates time. In addition, the first display region 41 is divided into a plurality of sub-regions 41A to 41D in the vertical direction. A first time axis 47 of the first display region 41 is provided above the first display region 41. The first time axis 47 is obtained by arranging markings and information, such as year, month, and day, according to the set time scale. The first time axis 47 has a length corresponding to the first display period of the first display region 41, and also has a width in the vertical direction so that information can be displayed therein. In this example, the first display period is set to about four months from October 2013 to early February 2014, and numbers indicating the year and month of four months corresponding to the first display period and markings attached at predetermined intervals between the respective months are displayed on the first time axis 47.

In the first display region 41, numerical data within the first display period of medical information is displayed in the form of graph G. As an examination image, a thumbnail image is displayed at a position corresponding to the imaging date. The first display period can be changed by the screen scrolling operation in the horizontal direction. By the screen scrolling operation, the display range of medical information is changed, and accordingly, the display of year and month of the first time axis 47 is also changed.

Medical information of a plurality of items acquired during the first display period is displayed in the sub-regions 41A to 41D. Accordingly, a plurality of items of the medical information in the same period are displayed so as to be able to be compared with each other. Medical information corresponding to display items set for each disease in the display item setting table 18 is displayed in the sub-regions 41A to 41D.

In this example, as examples of the display item of “disease A” designated in the disease designation portion 45, an item of dosing of drugs A and B is set for dosing•injection, items of blood pressure (top) and blood pressure (bottom) are set for vital signs, items of biochemical tests A and B are set for test substance examinations, and an item of CT examination is set for image examinations. The display items are displayed in the sub-regions 41A to 41D in order of dosing•injection, vital signs, test substance examinations, and image examinations. An attribute display column 48 regarding the display item is provided in each of the sub-regions 41A to 41D. In each attribute display column 48, major category names of display items such as “dosing•injection”, “vital”, “test substance examinations”, and “image examinations”, a middle category name such as “biochemical test”, or items such as “drugs A and B”, “blood pressure (top), blood pressure (bottom)”, “examination A, examination B”, and “CT examination” are displayed.

In the first sub-region 41A of the item of dosing•injection, a graph G showing a period for which the administration of each of the drugs A and B has been performed is displayed. In this example, since the dosages of both of the drugs A and B are fixed for the entire period, each graph G is displayed in the form of a bar graph that extends in a straight line in the horizontal direction. If the dosage is changed, the graph G is changed in the vertical direction. A display showing the numerical value (“100”, “50”) of the dosage is inserted in the graph G.

In the second sub-region 41B of the item of blood pressure, a graph G showing the transition of the measurement value of each of blood pressure (top) and blood pressure (bottom) is displayed. In the third sub-region 41C of the item of test substance examinations, a graph G showing the transition of the examination value of the test substance is displayed. In the fourth sub-region 41D, a thumbnail image of CT examinations is displayed.

In the first display region 41, screen scrolling in the vertical direction is also possible, so that the display range of each sub-region can be changed by the screen scrolling operation in the vertical direction. For example, when there is a fifth sub-region or the like that is not displayed on the display screen 15, the fifth sub-region or the like can be displayed by the screen scrolling operation in the vertical direction.

The second display region 42 is a region where a second time axis 49 having a relatively longer time scale than the first time axis 47 is displayed. The second time axis 49 indicates a second display period longer than the first display period, and a marking is displayed on the second time axis 49 annually, for example. A period indicator 51 is displayed on the second time axis 49. The period indicator 51 is an indicator showing to which period the first display period of the first display region 41 corresponds on the second time axis 49. The width of the period indicator 51 corresponds to the width of the first display period in the time scale of the second time axis 49. In this example, since the first display period is about four months, the width of the period indicator 51 corresponds to the width for about four months in the time scale of the second time axis 49.

The diagnostic assistance information display region 44 is a region where the diagnostic assistance information provided by the medical assistance server 11 is displayed. Since the diagnostic assistance information is calculated based on the medical information of a patient that corresponds to display items set for each disease, the content of the diagnostic assistance information is related to the display items of the first display region 41. In this example, diagnostic assistance information relevant to the biochemical test A that is one of display items, such as “examination value of the biochemical test A exceeds the standard value”, is provided.

A display item relevant to the provided diagnostic assistance information may be highlighted in the first display region 41. For example, since the diagnostic assistance information in this example is relevant to the examination value of the biochemical test A, the entire graph G of the biochemical test A in the first display region 41 or a portion exceeding the standard value in the graph G may be highlighted so as to be able to be distinguished from the other graphs G. As a highlighting method, there is a method of making the highlighted portion blink or a method of changing the color.

The diagnostic assistance information may include information regarding the treatment recommended based on the examination value in addition to the information regarding the diagnosis. Examples of the information regarding the recommended treatment include information of recommended drugs (“drug C”), side effect information on recommended drugs, and the like, as shown in this example.

The diagnostic assistance information display region 44 is provided on the display screen 15 where the medical information is displayed. Therefore, since the medical information and the diagnostic assistance information are displayed on one screen, it is easy to check such information.

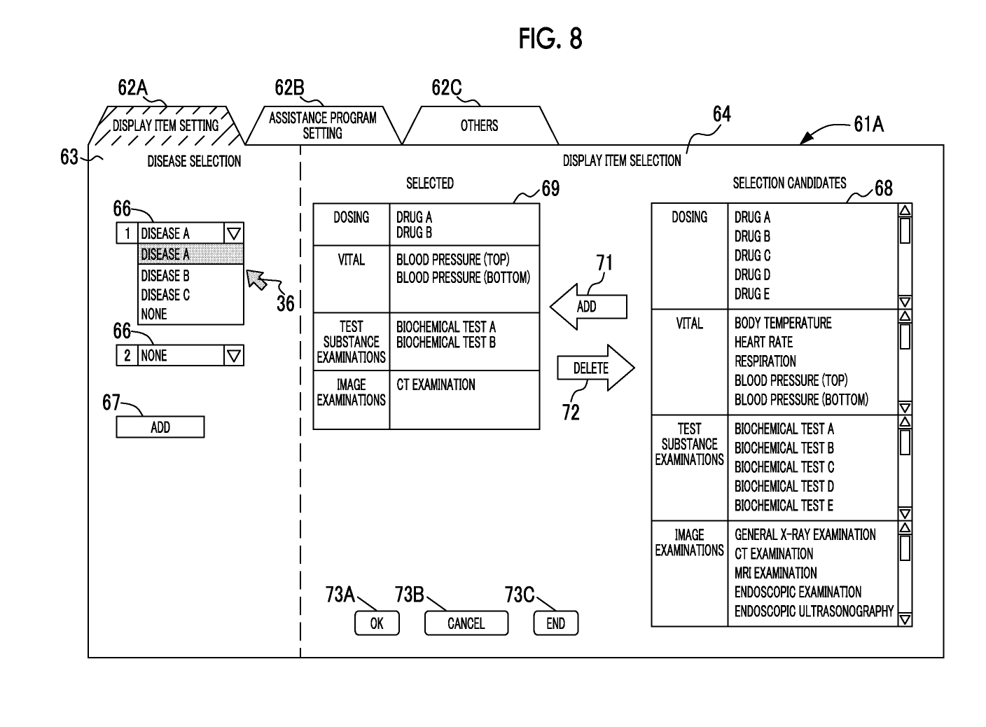

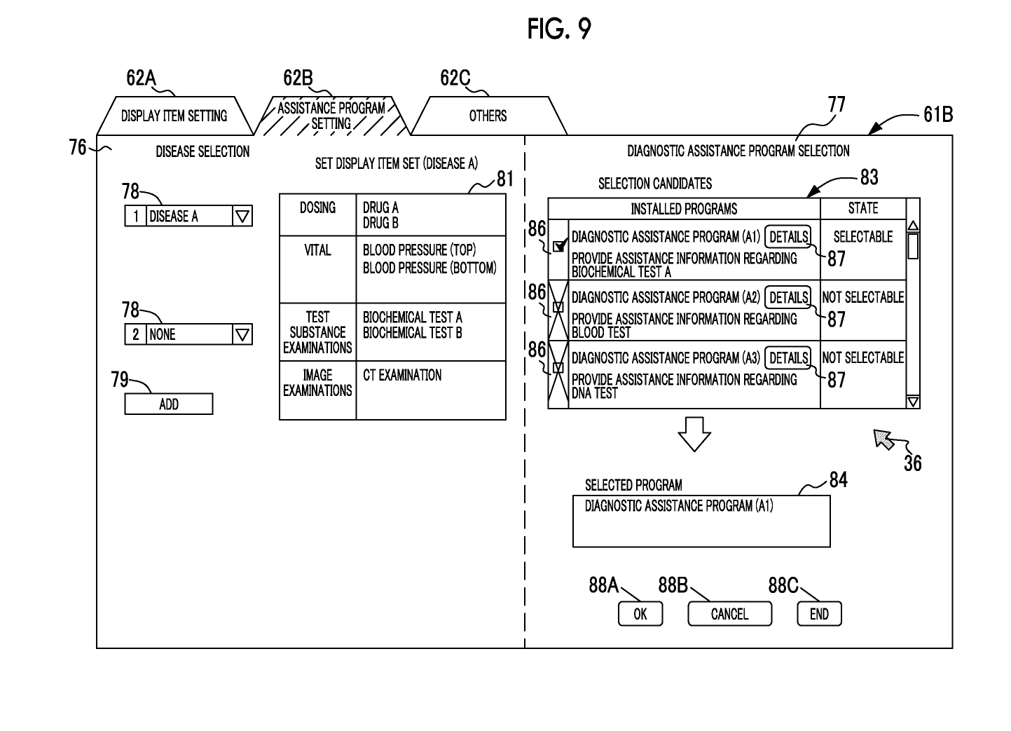

A setting key 52 is provided below the diagnostic assistance information display region 44. The setting key 52 is an operation button for performing a display item setting and an assistance program setting. When the setting key 52 is operated by the pointer 36, a setting screen 61 shown in FIGS. 8 and 9 is displayed. The setting screen 61 is a screen to enter an operation instruction for the display item setting and the assistance program setting. As described above, for the display item setting and the assistance program setting, setting authority is required. Therefore, when displaying the setting screen 61, authentication processing for checking whether or not the user is an administrator having setting authority is performed by requesting the input of a password or the like. In the setting screen 61, switching between a plurality of types of setting screens can be performed by tabs 62A to 62C, for example. When the tab 62A is selected by the pointer 36, a display item setting screen 61A shown in FIG. 8 is displayed.

A disease selection region 63 and a display item selection region 64 are provided on the display item setting screen 61A. A disease selection portion 66 is provided in the disease selection region 63. The disease selection portion 66 is an operation portion for selecting a disease as a group unit of medical care. The disease selection portion 66 is configured such that a disease list is displayed, for example, in a pull-down menu format, and a disease is selected from the disease list by the operation of the pointer 36.

A plurality of disease selection portions 66 are provided. Through the plurality of disease selection portions 66, it is possible to select multiple diseases when a patient has multiple diseases. In the example shown in FIG. 8, a state is shown in which “disease A” is selected in one disease selection portion 66 and “none” is selected in another disease selection portion 66. In this case, if, for example, “disease B” is selected instead of selecting “none”, it is possible to select multiple diseases of “disease A” and “disease B”. An add button 67 is an operation button for adding the disease selection portion 66. The add button 67 is used when selecting multiple diseases of three or more.

The display item selection region 64 is a region where display items corresponding to the disease selected in the disease selection region 63 are selected. A selection candidate display field 68 and a selected item display field 69 are provided in the display item selection region 64. The selection candidate display field 68 is a field for displaying the selection candidates of display items, and the selected item display field 69 is a field for displaying the selected display items that are selected from the selection candidate display field 68.

In the selection candidate display field 68, items as selection candidates are displayed for each major category of medical information, such as dosing, vital, test substance examinations, and image examinations. Items that are displayed in this selection candidate display field 68 are prepared in advance as initial values in a product (for example, the medical assistance server 11 or a program to generate the display item setting screen 61A). Alternatively, regardless of initial values, a doctor may perform editing, such as adding newly an item as a selection candidate or deleting all or some of initial values. When an add button 71 is operated in a state where a predetermined item in the selection candidate display field 68, for example, “drug A” is designated by the click operation of the pointer 36, the “drug A” is added to the selected item display field 69. On the contrary, when a delete button 72 is operated in a state where the added “drug A” is designated in the selected item display field 69, the “drug A” is deleted from the selected item display field 69. Through these operations, the selection of a display item is performed.

In addition, an OK button 73A, a cancel button 73B, and an end button 73C are provided in the display item selection region 64. The OK button 73A is a button to confirm the selection of a disease and a display item. When the OK button 73A is operated, the selection of a disease selected in the disease selection region 63 and a display item set including a plurality of display items selected in the selected item display field 69 is confirmed at the point in time that the operation occurs. When the selection is confirmed, a setting request including the selection information (display item selection information) of the disease and the display item set is transmitted to the medical assistance server 11, and the display item setting process is performed in the medical assistance server 11.

The end button 73C is an operation button to end the display item setting operation. When the end button 73C is operated, the setting screen 61 is closed. The cancel button 73B is an operation button to end the display item setting operation by canceling the input content.

As shown in FIG. 9, when the tab 62B is operated, an assistance program setting screen 61B is displayed. A disease selection region 76 and a diagnostic assistance program selection region 77 are provided in the assistance program setting screen 61B. A disease selection portion 78, an add button 79, and a set display item set display field 81 are provided in the disease selection region 76. The functions of the disease selection portion 78 and the add button 79 are the same as the functions of the disease selection portion 66 and the add button 67 of the display item setting screen 61A. In the set display item set display field 81, a display item set that has been set in the display item setting screen 61A is displayed according to the disease selected in the disease selection portion 78. In the example shown in FIG. 9, since the “disease A” is selected in the disease selection portion 78, a display item set that is set in association with the “disease A” in the display item setting screen 61A shown in FIG. 8 is displayed in the set display item set display field 81. When the disease selected in the disease selection portion 78 is changed, the display content of the set display item set display field 81 is also changed.

A selection candidate display field 83 of diagnostic assistance programs and a selected program display field 84 are provided in the diagnostic assistance program selection region 77. A plurality of diagnostic assistance programs for providing diagnostic assistance information regarding the disease selected in the disease selection region 76, among diagnostic assistance programs installed in the medical assistance server 11, are displayed in the selection candidate display field 83.

In FIG. 9, since the “disease A” is selected in the disease selection region 76, three diagnostic assistance programs A1 to A3 that provide diagnostic assistance information regarding the disease A are displayed. For example, the diagnostic assistance program A1 is intended to provide diagnostic assistance information regarding “biochemical test A”, the diagnostic assistance program A2 is intended to provide diagnostic assistance information regarding “blood test”, and the diagnostic assistance program A3 is intended to provide diagnostic assistance information regarding “DNA test”. In the selection candidate display field 83, it is possible to display diagnostic assistance programs other than the diagnostic assistance programs A1 to A3 by the scroll operation in the vertical direction.

In the selection candidate display field 83, selection availability information indicating whether or not selection is possible is displayed for each of the plurality of diagnostic assistance programs. In the medical assistance server 11, medical information corresponding to the display item is output as an operation parameter of the diagnostic assistance program. This is because optimal diagnostic assistance information should be able to be used within the range of display items set for each disease and a diagnostic assistance program for outputting the optimal diagnostic assistance information has processing conditions in which medical information corresponding to the display item that a doctor can view is used as an operation parameter and the content regarding the display item is output as diagnostic assistance information. That is, operation parameters used in a diagnostic assistance program and operation results are required to be within the range of display items. In the selection candidate display field 83, “selectable” is displayed for a diagnostic assistance program including such processing conditions, and “not selectable” is displayed for a diagnostic assistance program that does not include such processing conditions.

For example, the diagnostic assistance program A1 is a diagnostic assistance program that uses an item of “biochemical test A”, which is set as a display item for “disease A”, as a parameter and provides diagnostic assistance information regarding the “biochemical test A”. Therefore, the state of the diagnostic assistance program A1 in the selection availability information is “selectable”. On the other hand, the state of the diagnostic assistance program A2 or the diagnostic assistance program A3 that uses an item not corresponding to the display item for the “disease A” (“blood test” or “DNA test”) as a parameter or that provides diagnostic assistance information regarding an item not corresponding to the display item is “not selectable”.

A check box 86 is an operation portion for selecting a diagnostic assistance program. When the check box 86 is checked by the operation of the pointer 36, the checked diagnostic assistance program is selected. The selected diagnostic assistance program is displayed in the selected program display field 84. It is also possible to select a plurality of diagnostic assistance programs. For a diagnostic assistance program in a state of “not selectable”, the check box 86 cannot be checked either.

A detail button 87 is an operation button for displaying the detailed description of each diagnostic assistance program. For example, a detailed description regarding the function of each diagnostic assistance program or evidence information, such as medical knowledge that become the basis for the algorithm executed by the diagnostic assistance program, is displayed in the detailed description. The evidence information may include, for example, information indicating that medical knowledge as a basis has been established based on research on a certain population or information indicating that medical knowledge as a basis is affected by attributes of patients or a population, such as sex, age, and nationality. Since the evidence information can be easily checked by displaying such detailed description, it is easy to select a diagnostic assistance program based on the evidence information.

In the diagnostic assistance program selection region 77, an OK button 88A, a cancel button 88B, and an end button 88C are provided below the selected program display field 84. The functions of these buttons 88A to 88C are almost the same as the functions of the OK button 73A, the cancel button 73B, and the end button 73C of the display item setting screen 61A. The OK button 88A is a button to confirm the selection of a disease and a diagnostic assistance program. When the OK button 88A is operated, the selection of a disease selected in the disease selection region 76 and a diagnostic assistance program selected in the selected program display field 84 is confirmed at the point in time that the operation occurs. When the selection is confirmed, a setting request including the selection information (program selection information) of the disease and the diagnostic assistance program is transmitted to the medical assistance server 11, and the assistance program setting process is performed in the medical assistance server 11.

The end button 88C is an operation button to end the assistance program setting operation. When the end button 88C is operated, the setting screen 61 is closed. The cancel button 88B is an operation button to end the assistance program setting operation by canceling the input content.

When the display item setting process and the assistance program setting process are completed, display item sets and diagnostic assistance programs are set in association with respective diseases in the display item setting table 18, as shown in FIG. 10. In the example shown in FIG. 10, a display item set, which includes the items of “drugs A and B”, “blood pressure (top), blood pressure (bottom)”, “biochemical tests A and B”, and “CT examination”, and the diagnostic assistance program A1 are set in association with “disease A”. In addition, a display item set, which includes the items of “drugs A and C”, “blood pressure (top), blood pressure (bottom)”, “blood test E”, “biochemical test F”, and “endoscopic ultrasonography”, and two diagnostic assistance programs B2 and B6 are set in association with “disease B”.

In addition, a display item set, which includes the items of “drugs A, B, and C”, “blood pressure (top), blood pressure (bottom)”, “biochemical tests A, B, F, and G”, “blood test E”, “CT examination”, and “endoscopic ultrasonography”, and a diagnostic assistance program for multiple diseases AB7 are set in association with “multiple diseases AB” of the disease A and the disease B.

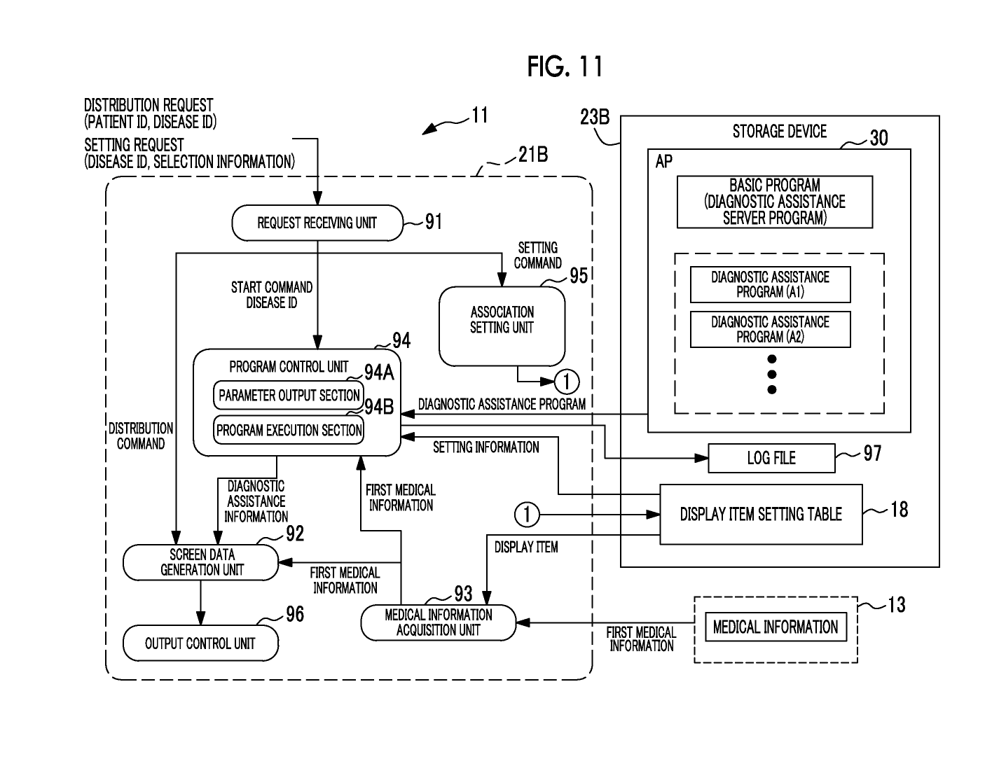

As shown in FIG. 11, a basic program that functions as a medical assistance server program (medical assistance program of the invention) is installed as the AP 30 in a storage device 23B of the medical assistance server 11. When the basic program is executed, a CPU 21B of the medical assistance server 11 cooperates with the memory 22 and the like to function as a request receiving unit 91, a screen data generation unit 92, a medical information acquisition unit 93, a program control unit 94, an association setting unit 95, and an output control unit 96.

A plurality of diagnostic assistance programs are installed in the storage device 23B. Each diagnostic assistance program is, for example, a module program that can be added or deleted, and is executed under the control of the program control unit 94. Since each diagnostic assistance program is a module program, updating or function addition can be easily performed. In the storage device 23B, the display item setting table 18 and a log file 97 to record the execution history of a diagnostic assistance program are stored.

The request receiving unit 91 receives a distribution request and a setting request from the client terminal 12. When the setting request is received, the request receiving unit 91 performs authentication processing for checking the setting authority of the requester. As described above, a display item setting request and an assistance program setting request are included in the setting request. The selected disease ID and display item selection information are included in the display item setting request. The selected disease ID and program selection information are included in the assistance program setting request. When the setting request is received, the request receiving unit 91 inputs a setting command based on the content of the setting request to the association setting unit 95. The association setting unit 95 updates the setting information of the display item setting table 18 according to the content of the setting command by performing the display item setting process and the assistance program setting process. Specifically, display items are set for each disease ID in the display item setting process, and the display items and diagnostic assistance programs are set in association with each disease ID in the assistance program setting process.

When a medical information distribution request is received, the request receiving unit 91 inputs a distribution command according to the content of the distribution request to the screen data generation unit 92. A patient ID and a disease ID that have been designated are included in the distribution request, and the request receiving unit 91 functions as a designation information receiving unit that receives designation information (group unit of medical care designation information) to designate the disease ID that is a group unit of medical care. By the input of the distribution command, the patient ID and the disease ID are input to the screen data generation unit 92. The screen data generation unit 92 requests the medical information acquisition unit 93 to acquire medical information by designating the input patient ID and disease ID.

The medical information acquisition unit 93 accesses the display item setting table 18 to read display items set for the designated disease ID. Then, the medical information acquisition unit 93 accesses the server group 13 to acquire medical information corresponding to the display items from the medical information of the designated patient ID. Here, the medical information corresponding to the display items is referred to as first medical information.

The medical information acquisition unit 93 outputs the acquired first medical information to the screen data generation unit 92 and the program control unit 94. The screen data generation unit 92 generates screen data of the display screen 15 based on the input first medical information. The output control unit 96 distributes the screen data generated by the screen data generation unit 92 to the requesting client terminal 12 through the network 14. Accordingly, the first medical information is distributed to the client terminal 12.

On the other hand, when a distribution request is received, the request receiving unit 91 inputs a start command including the disease ID to the program control unit 94. When the start command is received, the program control unit 94 executes a diagnostic assistance program and outputs the diagnostic assistance information. The program control unit 94 includes a parameter output section 94A and a program execution section 94B, and controls the starting of a diagnostic assistance program and the output of parameters for a diagnostic assistance program to be started.

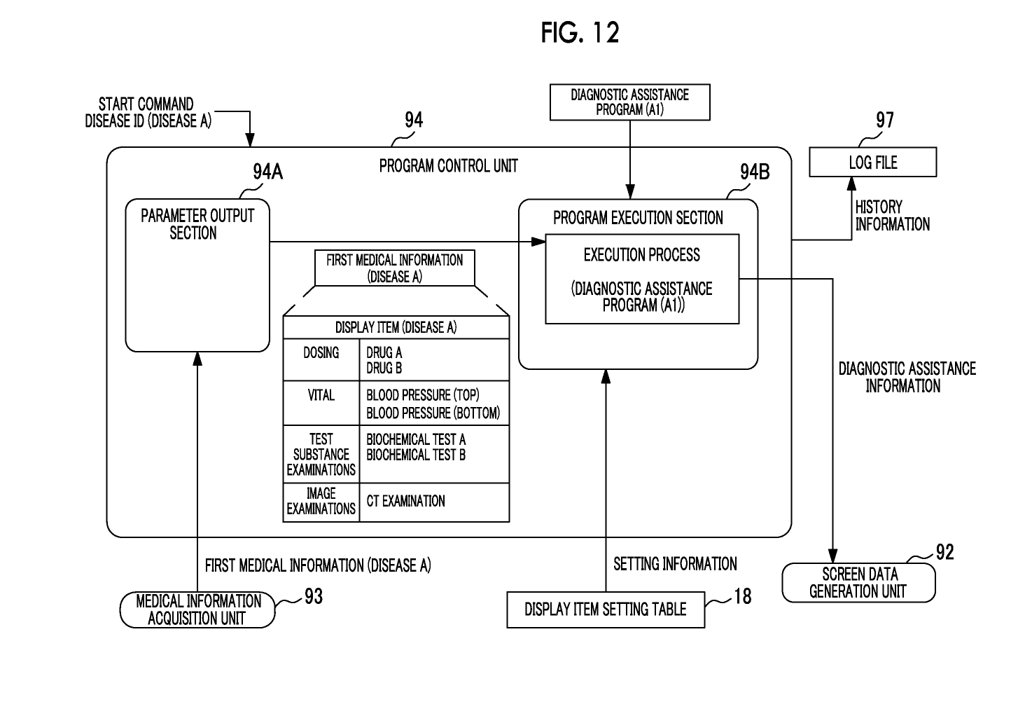

As shown in FIG. 12, first, the program control unit 94 determines a diagnostic assistance program, which is associated with the disease ID designated in the start command, to be a diagnostic assistance program to be started with reference to the setting information of the display item setting table 18. Then, the determined diagnostic assistance program is read from the storage device 23B. For example, when “disease A” is designated in the start command, the diagnostic assistance program A1 is set so as to be associated with the “disease A” in the display item setting table 18. The program control unit 94 determines the diagnostic assistance program A1 to be a diagnostic assistance program to be started.

The program execution section 94B starts the read diagnostic assistance program A1 to generate an execution process. The first medical information regarding the “disease A” that has been acquired by the medical information acquisition unit 93 is input to the parameter output section 94A. The parameter output section 94A outputs all pieces of the first medical information regarding the “disease A”, as parameters to be input to the diagnostic assistance program A1, to the program execution section 94B in a form in which it is possible to identify that these are display items. In this example, the parameter output section 94A outputs only the first medical information that is set as display items. Therefore, the program execution section 94B can determine that all parameters output from the parameter output section 94A are display items. Thus, in the present embodiment, outputting only the first medical information is defined as an output in the identifiable form. The program execution section 94B calculates diagnostic assistance information by performing an operation according to the execution process of the diagnostic assistance program A1 with the first medical information as parameters.

The calculated diagnostic assistance information is output to the screen data generation unit 92. The screen data generation unit 92 generates screen data of the display screen 15 on which the diagnostic assistance information is inserted in the diagnostic assistance information display region 44. The output control unit 96 distributes the screen data generated by the screen data generation unit 92 to the requesting client terminal 12 through the network 14. Accordingly, the diagnostic assistance information is distributed to the client terminal 12.

When the diagnostic assistance program is executed, the program execution section 94B records history information regarding the execution in a log file 97. Date and time in which the diagnostic assistance program was executed, a patient ID or a disease ID regarding the first medical information used as an operation parameter, display item names included in the first medical information, and the like are included in the history information.

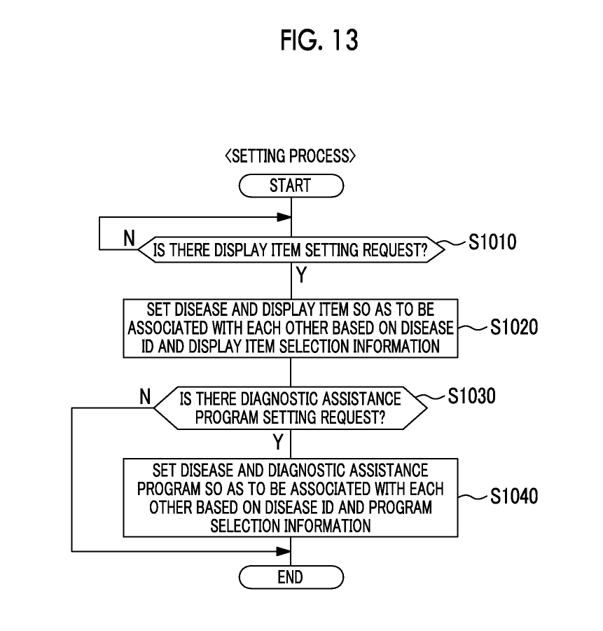

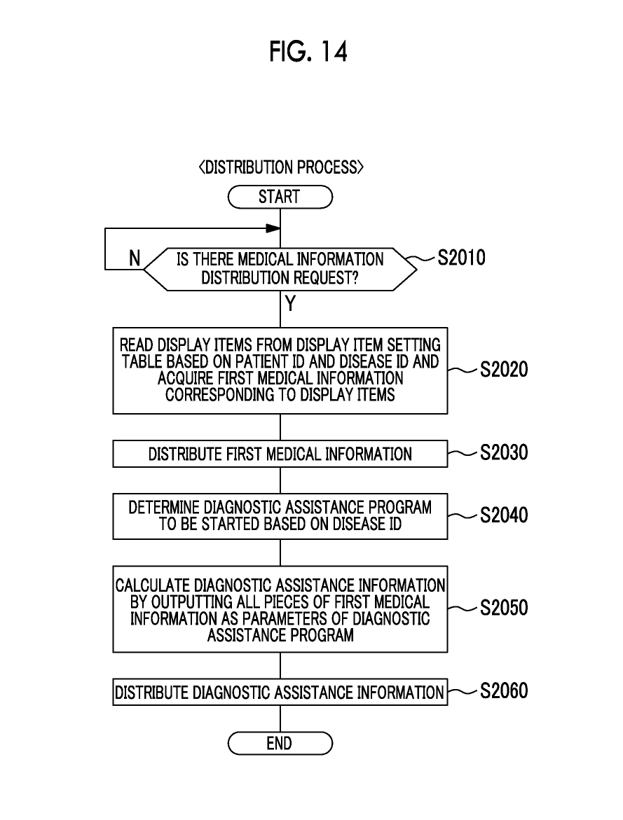

Hereinafter, the operation of the above configuration will be described with reference to FIGS. 13 and 14. When performing a display item setting or an assistance program setting, a setting request is issued through the setting screen 61 (FIGS. 8 and 9) of the client terminal 12 by the setting operation of an administrator having setting authority.

As shown in FIG. 13, when a display item setting request is received (Y in S1010), the medical assistance server 11 performs a display item setting process for performing a setting by associating the display item setting table 18 with a disease and a display item based on the disease ID and the display item selection information included in the setting request (S1020). In addition, when a diagnostic assistance program setting request is received (Y in S1030), the medical assistance server 11 performs an assistance program setting process for performing a setting by associating a disease with a diagnostic assistance program based on the disease ID and the program selection information included in the setting request (S 1040). Accordingly, display items and diagnostic assistance programs are set so as to be associated with each disease.

When viewing the medical information, a doctor designates a patient ID and a disease ID and issues a distribution request using the client terminal 12. When the distribution request is received (Y in S2010), the medical assistance server 11 reads display items from the display item setting table 18 based on the disease ID included in the distribution command. Then, first medical information corresponding to the display items of the medical information of the designated patient ID is acquired from the server group 13 (S2020).

The screen data generation unit 92 generates screen data of the display screen 15 on which the first medical information is displayed. The generated screen data is distributed to the client terminal 12 by the output control unit 96 (S2030). Based on the disease ID, the program control unit 94 determines a diagnostic assistance program to be started with reference to the display item setting table 18 (S2040). The program control unit 94 starts the determined diagnostic assistance program, and generates the execution process of the diagnostic assistance program in the program execution section 94B.

The parameter output section 94A outputs the first medical information input from the medical information acquisition unit 93, as parameters of the diagnostic assistance program, to the program execution section 94B. The program execution section 94B calculates diagnostic assistance information by performing an operation according to the execution process of the diagnostic assistance program with the first medical information as parameters (S2050). The calculated diagnostic assistance information is distributed to the client terminal 12 through the screen data generation unit 92 and the output control unit 96 (S2060).

As described above, in the present embodiment, there is provided the association setting unit 95 that sets display items displayed on the display screen 15 viewed by the doctor, among the pieces of medical information of the patient, and diagnostic assistance programs so as to be associated with each disease. Therefore, display items to be used for diagnosis and a diagnostic assistance program to be started according to the display items can be determined for each disease. If a diagnostic assistance program suitable for diagnosis is determined for each disease, a diagnostic assistance program suitable for diagnosis is automatically selected by the designation of a disease. As a result, compared with a case of searching for a diagnostic assistance program to be started each time, working efficiency is improved.

In the present embodiment, there is provided the parameter output section 94A that outputs all pieces of the first medical information corresponding to display items as parameters in a form in which it is possible to identify that these are display items. Therefore, since it is possible to check all the display items determined for each disease based on the individual circumstances of the medical field, such as medical facilities or departments, the diagnostic assistance program can provide optimal diagnostic assistance information available in the range of the display items. In other words, since the diagnostic assistance program can check all pieces of the first medical information corresponding to display items, it is possible to maximize the function of the diagnostic assistance program in the range of the first medical information by using the first medical information effectively. Therefore, it is possible to provide optimal diagnostic assistance information. If a diagnostic assistance program created based on the latest medical knowledge is used as the diagnostic assistance program, it is also possible to provide the latest medical knowledge in the range of the first medical information.

In addition, since a parameter output section that outputs all pieces of the first medical information corresponding to display items is provided, an interface to output the first medical information in the medical assistance server 11 is clarified. Therefore, since program development companies are likely to participate in the development of diagnostic assistance programs, it is possible to develop diagnostic assistance programs efficiently. In addition, if a system is adopted in which information regarding the types of parameters acquired by the diagnostic assistance program is uploaded online (through a network) to a server of a developer using the log file 97 or the like, it becomes easy to check the first medical information in each hospital. As a result, it is possible to provide a development environment in which improvements in the diagnostic assistance program capable of providing the optimal diagnostic assistance information in the range of the first medical information can be efficiently made.

In addition, the first medical information is subject to change daily depending on the establishment of new medical knowledge or the individual circumstances of the medical field. In the medical field, it is possible to easily change the content of the first medical information using the association setting unit. On the other hand, since the parameter output section outputs all pieces of the first medical information even when the first medical information is changed, it becomes easy for the developer of the diagnostic assistance program to check the change of the first medical information. Therefore, it also becomes easy to perform continuous improvements in the diagnostic assistance program or the addition of a new function according to the change of the first medical information.

As a general development form of a diagnostic assistance program in the past, if medical knowledge has been established, parameters for effectively using the medical knowledge have been determined first and then the diagnostic assistance program has been developed in many cases. That is, the diagnostic assistance program has been developed, in many cases, without a focus on whether or not parameters used in the diagnostic assistance program can be used in each medical field. It cannot be said that even a diagnostic assistance program based on the latest medical knowledge is useful if the diagnostic assistance program cannot be used in the medical field.

In contrast, according to the present embodiment, since a parameter output section is provided, it is easy to perform development giving first priority to using the first medical information corresponding to display items, which can be used in the medical field, as parameters. As a result, it is possible to provide a diagnostic assistance program that can be easily used in the medical field.

In addition, as the accumulation of big data for medical information further proceeds, the number of items of medical information of one patient is also expected to increase. In this case, there is a high possibility that individual differences in the number of items will occur. For example, there is an item A in the medical information of a certain patient, but there is no item A in the medical information of other patients. Specifically, there is a deoxyribonucleic acid (DNA) test that has become easier to use recently. Although research to provide optimal medical treatment by DNA information is being actively done, it depends on an individual whether or not to have a DNA test. Accordingly, individual differences in the number of items occur. Even in such a case, it is necessary to provide the optimal assistance for both a person with DNA information and a person with no DNA information. Therefore, in the future, the diagnostic assistance program is expected to be developed considering predetermined individual differences in the number of items so that it is possible to flexibly respond to various circumstances. For example, the diagnostic assistance program is expected to flexibly respond to changes in the number of items. Specifically, it is expected that the diagnostic assistance program can perform an operation with an alternative item even if a certain item is not present. On the contrary, it is expected that the diagnostic assistance program can use the increased items effectively when the number of items has increased.

When the display item setting table 18 is changed, the number of items of the first medical information is also changed. Even if the number of items of the first medical information is changed, the parameter output section 94A is configured to output all pieces of the first medical information at that point in time. If the parameter output section 94A is combined with a diagnostic assistance program that can flexibly respond to changes in the number of items, the diagnostic assistance program can flexibly respond to changes in the number of items of the first medical information output from the parameter output section 94A. As a result, since it is possible to provide the optimal diagnostic assistance information corresponding to the latest first medical information, a synergistic effect can be expected.

In this example, the first medical information including examination images has been described as an example. However, when outputting examination images as a parameter, all of the examination images may be output as a parameter, or some of the examination images may be output as a parameter, for example. For example, when there are examination images of a plurality of modalities, only the latest examination image of each modality may be output as a parameter. In addition to the examination image itself, image analysis information (lesion size or the like) obtained by analyzing the examination image may be output as a parameter as a set. The lesion size indicates the size of a tumor, for example. Therefore, it is possible to calculate appropriate diagnostic assistance information by using the lesion size. In addition, it is also possible to select an examination image to be output as a parameter using the lesion size. For example, an examination image having a lesion size equal to or greater than a predetermined value may be output as a parameter. For example, blood flow measurement using an ultrasonic wave is included in the image analysis information.

In this example, the case where one disease is designated in the distribution request has been described as an example. However, when multiple diseases are designated, it is preferable that the program control unit 94 preferentially starts a diagnostic assistance program for multiple diseases (for example, the diagnostic assistance program AB7 shown in FIG. 10) that is suitable for multiple diseases. Multiple diseases are more common in the elderly. As aging becomes faster in recent years, the number of patients suffering from multiple diseases is also increasing. For this reason, such a function is effective.

In this example, history information regarding the execution of a diagnostic assistance program is recorded in the log file 97. Therefore, medical information input as a parameter can also be checked by the log file 97. Since the log file 97 can be read, for example, online as described above or during maintenance of the diagnostic assistance program, it is possible to easily check the parameters output from the parameter output section 94A. This is very convenient for development companies of diagnostic assistance programs.

In addition, it is preferable that the parameter output section 94A standardizes a data format to output the first medical information.

### Second Embodiment

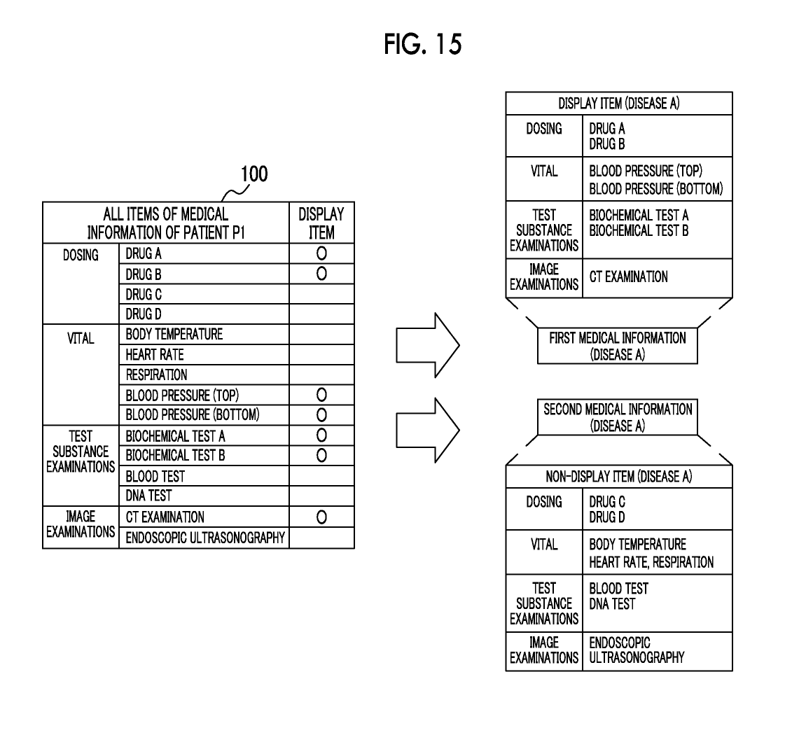

A second embodiment shown in FIGS. 15 to 17 is an example in which the parameter output section 94A can output second medical information not corresponding to display items, as a parameter of a diagnostic assistance program, in addition to the first medical information corresponding to display items. As shown in FIG. 15, for example, all items of medical information of patients that can be acquired from the server group 13 are recorded in a medical information table 100. FIG. 15 illustrates the medical information of a “patient P1”. In the medical information table 100, circles are given to the first medical information corresponding to the display item regarding “disease A”, for example. The second medical information is all of the remaining items after excluding the first medical information from all the items. As shown in FIG. 15, not only examination values or measurement values but also examination images of endoscopic ultrasonography and the like may be included in the second medical information. When a patient has regular checkups, new symptoms or the like may occur in the middle of treatment. In this case, since a display item needs to be added, it is also possible to add a display item at any time. For the addition of a display item, not only is the addition based on selection from selection candidates prepared in advance possible, but also a new item, which is not included in the selection candidates prepared in advance, can be added arbitrarily.

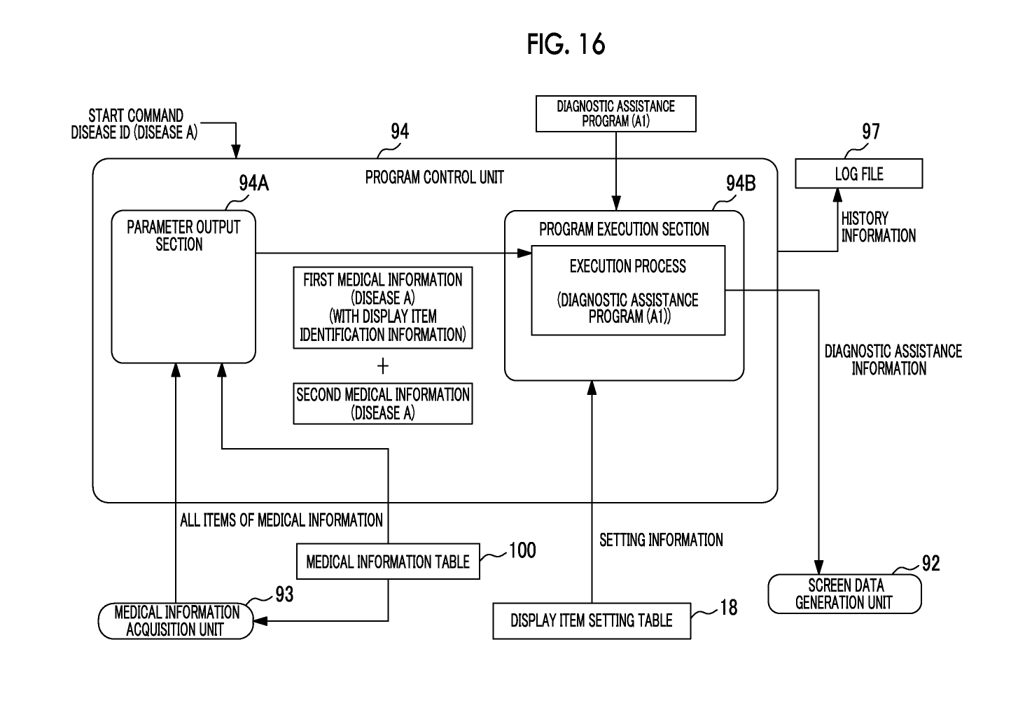

As shown in FIG. 16, in the second embodiment, when there is a distribution request, the medical information acquisition unit 93 acquires all the items of the medical information from the server group 13 with reference to the medical information table 100. Then, the acquired medical information of all the items is input to the parameter output section 94A. The parameter output section 94A determines whether each item of the input medical information is the first medical information corresponding to display items or the second medical information not corresponding to display items with reference to the medical information table 100. The parameter output section 94A gives identification information for identifying that each item of the input medical information is a display item to the first medical information, and outputs the result to the program execution section 94B. The second medical information is output to the program execution section 94B without identification information being given thereto. Accordingly, the first medical information is output in a form in which it is possible to identify that the first medical information is display items. In the present embodiment, outputting the first medical information with the identification information given thereto is defined as an output in the identifiable form. As the identification information, it is possible to use any form, such as an identification flag and an item name.

The program execution section 94B identifies the first medical information and the second medical information based on the identification information given to the first medical information. The program execution section 94B calculates diagnostic assistance information through the operation with the first medical information as parameters. In addition, the program execution section 94B can use the second medical information for the calculation of diagnostic assistance information.

As the method of use of the second medical information, for example, the following two methods can be considered. The first method of use is a method of using the second medical information as a parameter for improving the calculation accuracy of diagnostic assistance information when the content of diagnostic assistance information to be calculated is limited to the range of the first medical information. Since the content of the diagnostic assistance information to be calculated is limited to the range of the first medical information and accordingly is in the viewable range of the doctor, it is easy to use the diagnostic assistance information.

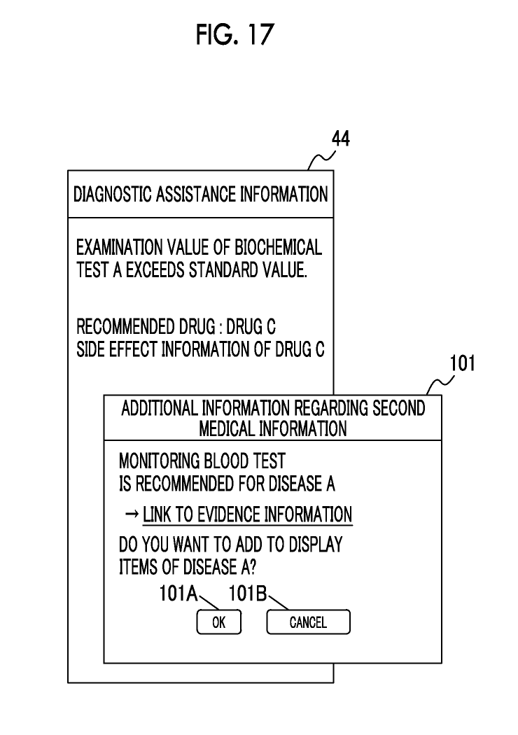

The second method of use is a method of calculating diagnostic assistance information in which not only the content regarding the first medical information but also the content regarding the second medical information is included. The diagnostic assistance information in this case is shown in FIG. 17, for example. In FIG. 17, the content regarding the first medical information is the same as the content shown in FIG. 7, and the content regarding “biochemical test A” corresponding to a display item is provided in the diagnostic assistance information display region 44. On the other hand, as the content regarding the second medical information, for example, information indicating “monitoring the blood test is recommended for the disease A” is provided. The content regarding the second medical information is displayed in a sub-display field 101, so that it can be seen that the content regarding the second medical information is additional information.

An item not corresponding to display items regarding “disease A”, such as “blood test” in this example, may become an item that should be noted in the diagnosis according to the latest medical knowledge. Therefore, the second method of use can help provide a doctor with the latest medical knowledge to which the doctor has not paid attention. In the second method of use, it is preferable to display evidence information, which indicates the medical basis for additional information to be provided, in the sub-display field 101 or to display at least a link for accessing the evidence information.

As shown in FIG. 17, it is also preferable to display a message (“Do you want to add to the display items of the disease A?”) prompting the user to newly add the item regarding the second medical information, which is provided as additional information, to the display items. In this case, it is preferable to provide an OK button 101A or a cancel button 101B in the sub-display field 101 so that it is possible to select whether or not to set the recommended item in the display items in the sub-display field 101. When the OK button 101A is operated, for example, a setting request is transmitted to the medical assistance server 11, and the display item setting table 18 is updated. Needless to say, the update of the display item setting table 18 is not allowed if there is no setting authority. However, even in such a case, it is preferable to transmit a message indicating addition to the display items to the administrator having setting authority by e-mail, for example. In addition, the number of items of each of the first medical information and the second medical information that are provided as diagnostic assistance information may be two or more.

### Third Embodiment

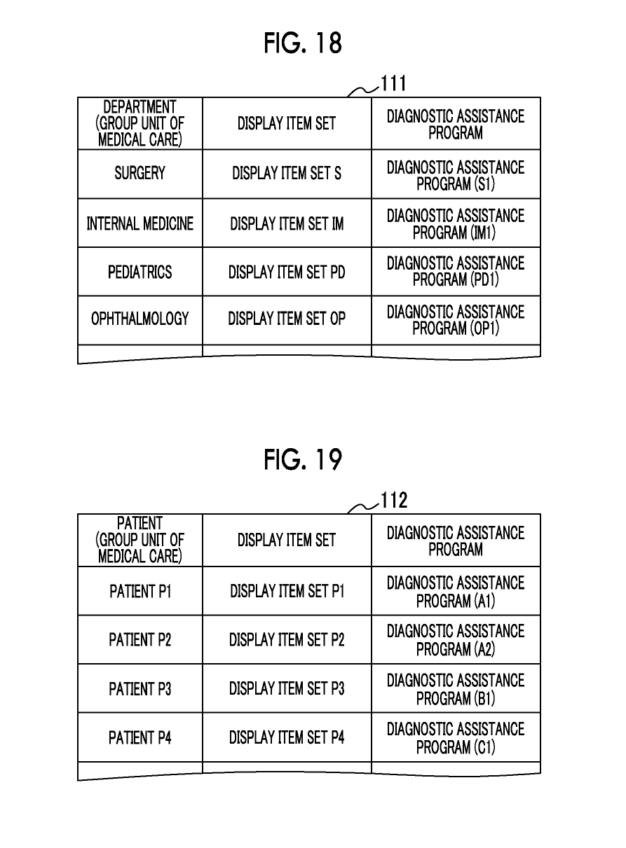

In the first and second embodiments, a disease has been described as an example of the group unit of medical care. However, the group unit of medical care is not limited to the disease, and it is possible to use various kinds of group unit of medical cares as in a third embodiment shown in FIGS. 18 to 21. In a display item setting table 111 shown in an example of FIG. 18, the group unit of medical care is a department, such as surgery, internal medicine, pediatrics, and ophthalmology. If the department is different, medical equipment is different. Accordingly, examination values or measurement value that are acquired are also different, and items (examination values or measurement value) of medical information to be noted are also different. In the display item setting table 111, a display item set corresponding to the department is set, and the set display item set and a diagnostic assistance program are set so as to be associated with each department.

In a display item setting table 112 shown in an example of FIG. 19, the group unit of medical care is a patient. Since the item of medical information to be noted changes according to a patient, the display item is also changed. In the display item setting table 112, a display item set corresponding to the patient is set, and the set display item set and a diagnostic assistance program are set so as to be associated with each patient.

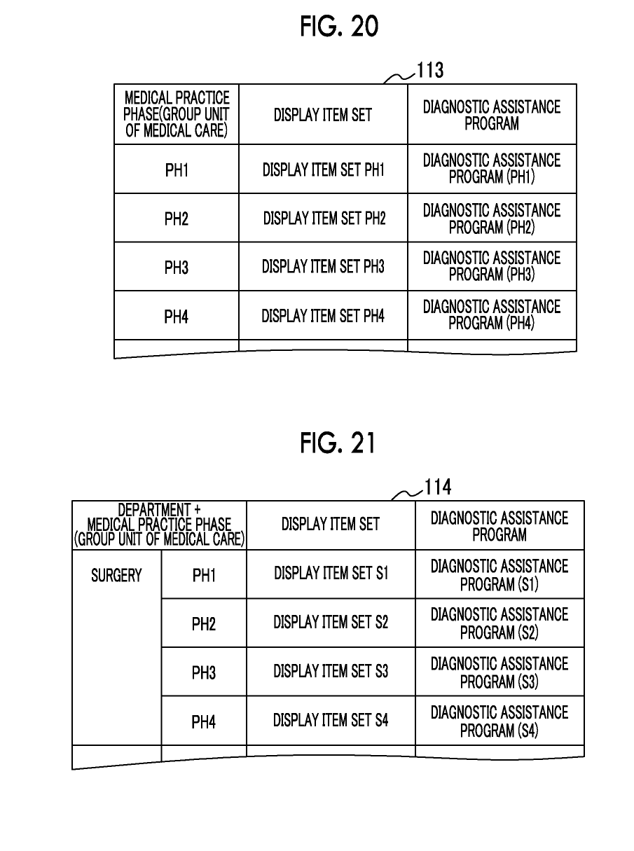

In a display item setting table 113 shown in an example of FIG. 20, the group unit of medical care is a medical practice phase that is a progress phase of medical treatment or disease. If the medical practice phase is different, the item of medical information to be noted is also changed. In the display item setting table 113, a display item set corresponding to the medical practice phase is set, and the set display item set and a diagnostic assistance program are set so as to be associated with each medical practice phase.

In addition, as in a display item setting table 114 shown in FIG. 21, a plurality of group unit of medical cares, such as a department and a medical practice phase, may be combined. In this case, a display item set is set for each combination of a plurality of group unit of medical cares, and the set display item set and a diagnostic assistance program are set so as to be associated with each other.

### Fourth Embodiment

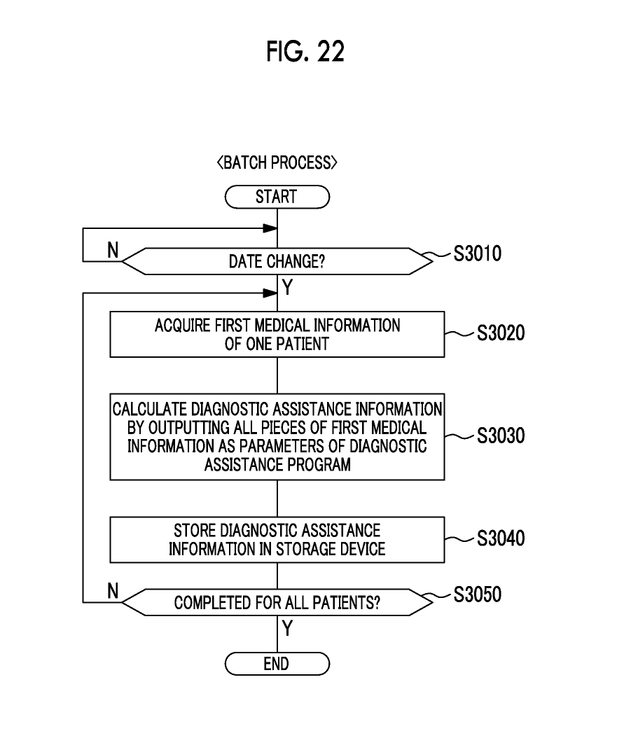

In the first embodiment, an example has been described in which the diagnostic assistance information of one person is calculated whenever a distribution request for medical information of one patient is received. However, a fourth embodiment shown in FIG. 22 is an example in which the program control unit 94 starts a diagnostic assistance program and performs a batch process for collectively calculating the diagnostic assistance information of a plurality of patients. By performing the batch process, it is possible to shorten the response time of diagnostic assistance information to the client terminal 12.

As shown in the flowchart of FIG. 22, the batch process is performed at the timing when the date is changed, for example. When the date is changed (Y in S3010), the medical assistance server 11 acquires the first medical information of one patient (S3020). Then, diagnostic assistance information is calculated by outputting all pieces of the acquired first medical information as parameters of the diagnostic assistance program (S3030). Then, the calculated diagnostic assistance information is stored in the storage device 23B (S3040). After the process for one person ends, the above process is performed for the next patient. Such a process is repeated for all patients (N in S3050). This process is ended when the process has been completed for all patients (Y in S3050).

### Fifth Embodiment

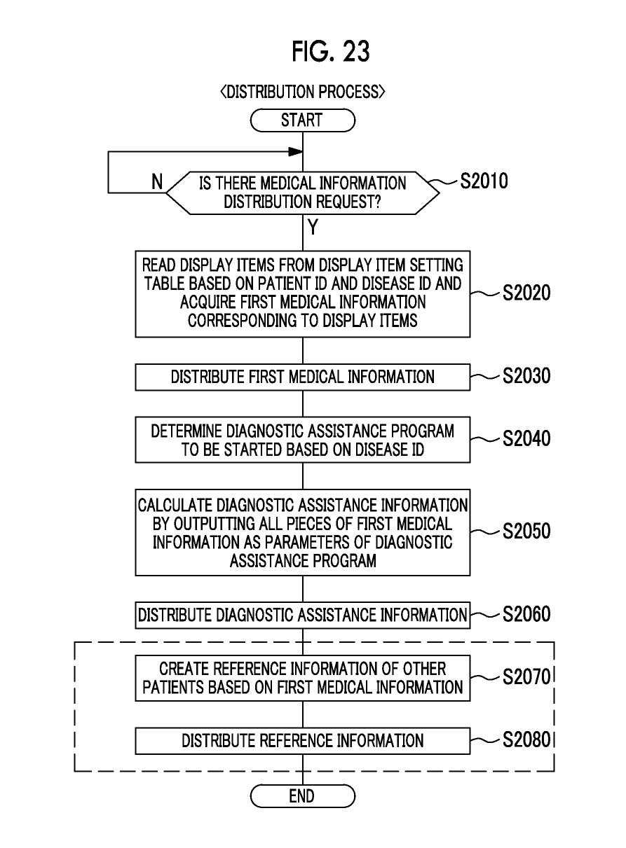

A fifth embodiment shown in FIG. 23 is an example in which, when providing diagnostic assistance information regarding one patient, reference information of other patients is created and the created reference information is distributed. The flowchart in the fifth embodiment is the same as the flowchart shown in FIG. 14 in the first embodiment up to S2060, but a process of creating reference information of other patients based on the first medical information (S2070) and a process of distributing the reference information (S2080) are different. Hereinafter, differences will be described.

Reference information is, similar cases of other patients having examination values or measurement values close to those of a patient of interest or having similar symptoms, for example. The medical assistance server 11 searches for a similar case from the server group 13 based on the first medical information of the patient of interest, for example. As similar symptoms, for example, information of patients indicating similar examination values for the same examination item is searched. Information indicating what drugs were effective for the patients or what drugs were not effective for the patients, and the like is created as reference information. For example, regarding whether or not there is a variation in the efficacy of the same drug according to the attributes (sex, age, nationality, and the like) of a patient, a histogram may be created for each attribute and the histogram may be provided as reference information. If such reference information is created using the items of the first medical information, such reference information can be easily compared with the first medical information of the patient of interest.

### Sixth Embodiment

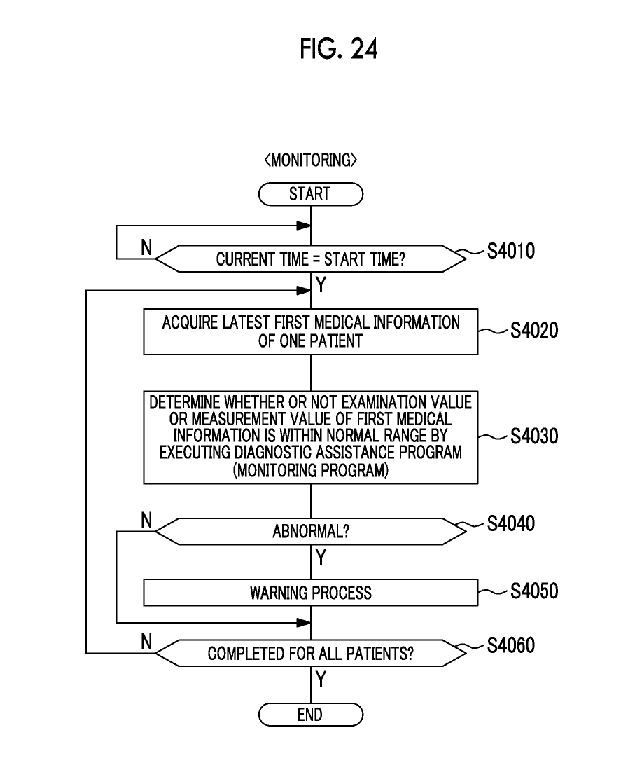

A sixth embodiment shown in FIG. 24 is an example in which a monitoring program is periodically started as a diagnostic assistance program in order to monitor the first medical information. The monitoring program has a monitoring function of monitoring whether or not the examination value or the measurement value is within a normal range for one or more items of the first medical information. The program control unit 94 executes the monitoring program periodically.

For example, as shown in the flowchart of FIG. 24, when the current time periodically reaches the start time (Y in S4010), the program control unit 94 starts the monitoring program. First, the program control unit 94 acquires the latest first medical information of one patient through the medical information acquisition unit 93 (S4020). The first medical information is output to the program execution section 94B from the parameter output section 94A. The program execution section 94B determines whether or not the examination value or the measurement value of the first medical information is within a normal range by executing the monitoring program (S4030). When it is determined that the examination value or the measurement value of the first medical information is within the normal range (S4040) and there is the latest first medical information of the next patient (N in S4060), the program execution section 94B acquires the latest first medical information of the next patient and determines whether or not the examination value or the measurement value of the latest first medical information is within a normal range.

When the examination value or the measurement value is out of the normal range, the program execution section 94B determines “abnormal” (Y in S4040), and performs a warning process (S4050). The warning process is performed, for example, by sending an e-mail to a family doctor of the patient who is a warning target. In the case of determination of “abnormal”, if there is a distribution request from the client terminal 12 for the medical information of the patient who is a warning target, the medical assistance server 11 may transmit warning information to the requesting client terminal 12. In this case, the warning information is displayed on the display screen 15. Alternatively, when a first distribution request for the medical information of the patient who is a warning target is received after the determination of “abnormal”, warning information may be distributed to the client terminal 12. Thus, by periodically monitoring whether or not the examination values or the measurement values in the latest medical information are within the normal range, it is possible to send a warning for abnormalities early. The above process is repeated for all patients (S4060).

### Seventh Embodiment

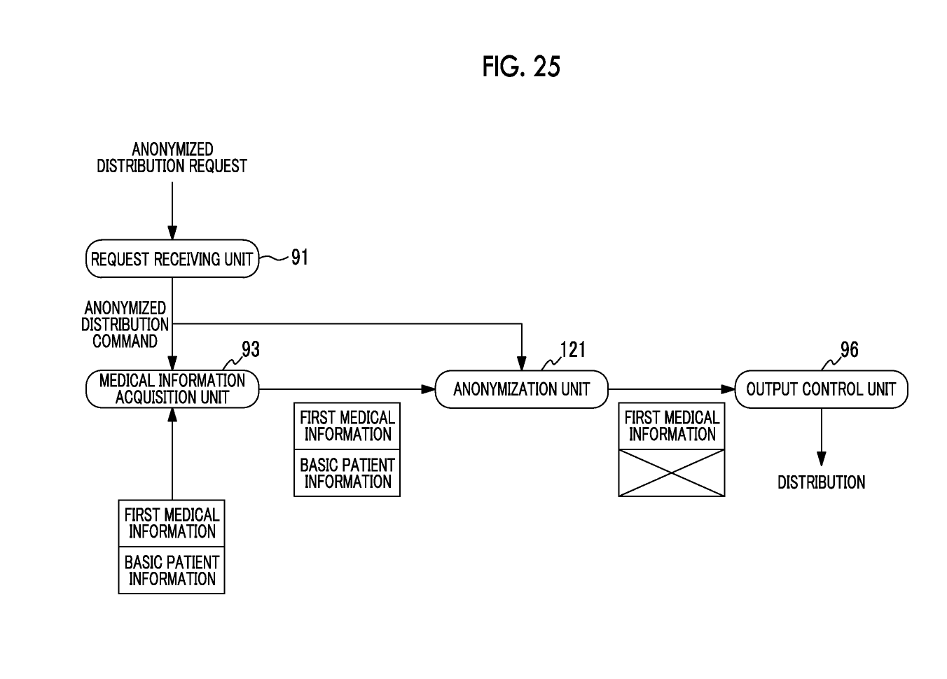

As in a seventh embodiment shown in FIG. 25, an anonymization unit 121 that anonymizes the personal information of a patient in the first medical information is provided. The anonymization unit 121 anonymizes the first medical information by deleting personal information, such as basic patient information, from the first medical information acquired by the medical information acquisition unit 93. When acquiring the anonymized first medical information, for example, an anonymized distribution request is transmitted to the medical assistance server 11. The request receiving unit 91 inputs an anonymized distribution command to the medical information acquisition unit 93 and the anonymization unit 121 based on the anonymized distribution request. The anonymization unit 121 distributes the anonymized first medical information to the requester through the output control unit 96. In addition to the personal information, information of medical facilities may be anonymized. Thus, by providing the anonymization unit 121, it becomes easy to provide development companies of diagnostic assistance programs or the like with the first medical information used in the development of diagnostic assistance programs. Therefore, the medical facilities can serve as main agents to develop diagnostic assistance programs required therefor. In addition, this is also convenient when passing the first medical information to an external service company to request an analysis service or the like.

### Eighth Embodiment

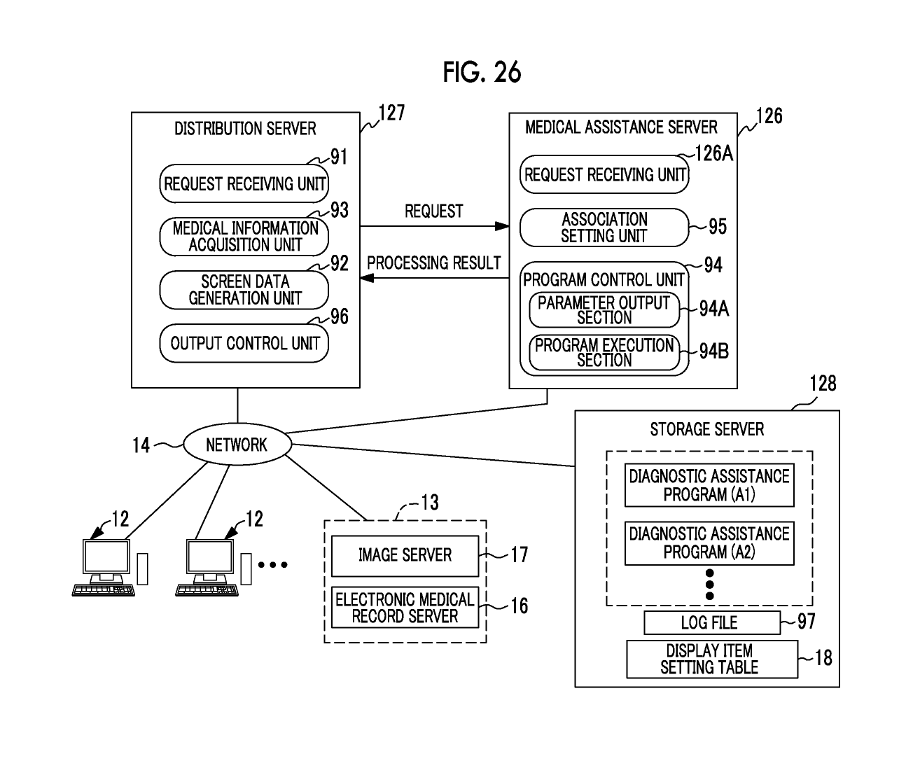

In each of the above embodiments, the medical assistance device of the invention has been described in the form of the medical assistance server 11 having a screen distribution function of distributing screen data of the display screen 15, on which medical information is displayed, based on the request from the client terminal 12. However, another server may be made to have a screen distribution function, as a medical assistance server 126 shown in FIG. 26.

The medical assistance server 126 shown in FIG. 26 does not have a screen distribution function, and includes a request receiving unit 126A, an association setting unit 95, and a program control unit 94 including a parameter output section 94A and a program execution section 94B. The request receiving unit 126A receives a processing request, such as diagnostic assistance information calculation processing, from a distribution server 127. A request receiving unit 91, a screen data generation unit 92, a medical information acquisition unit 93, and an output control unit 96 are provided in the distribution server 127. The request receiving unit 91 receives a distribution request or a setting request from the client terminal 12. When a distribution request is received, the request receiving unit 91 inputs a distribution command to the screen data generation unit 92. When a setting request is received, the request receiving unit 91 transmits a request for setting process to the medical assistance server 126.

A diagnostic assistance program, a display item setting table 18, and a log file 97 may be stored in an external storage server 128 provided separately from the medical assistance server 126 or the distribution server 127. The medical assistance server 126 or the distribution server 127 accesses the storage server 128 to acquire various kinds of information when necessary.

### Ninth Embodiment

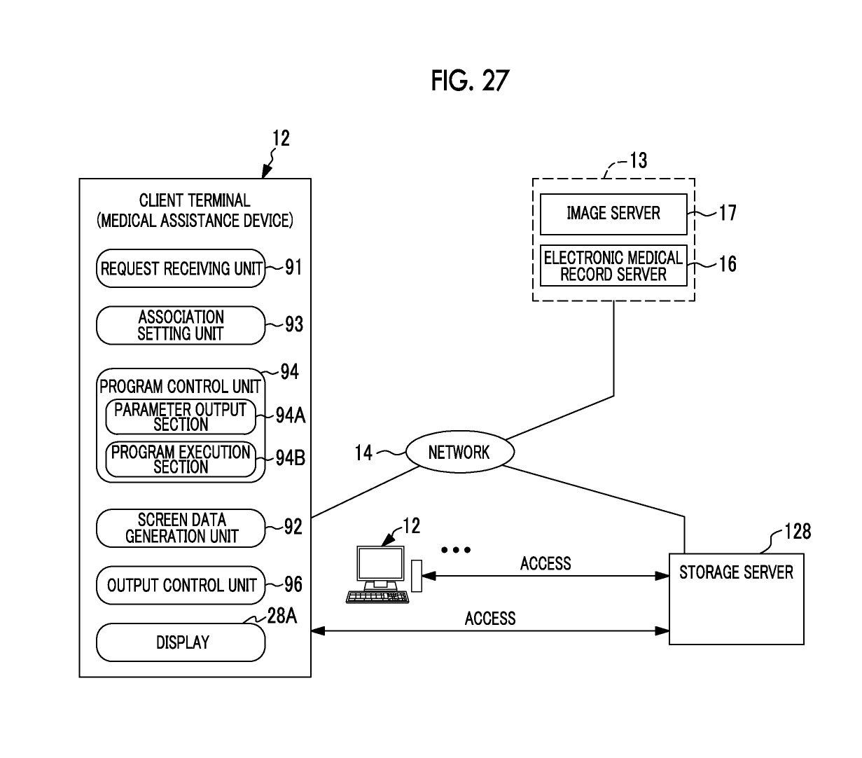

As shown in FIG. 27, instead of the medical assistance servers 11 and 126, a client terminal 12 may be made to serve as a medical assistance device of the invention. In this case, a request receiving unit 91, an association setting unit 95, a program control unit 94, a screen data generation unit 92, and an output control unit 96 are provided in the client terminal 12. The client terminal 12 accesses a server group 13 or a storage server 128 to acquire the required information, such as medical information or a diagnostic assistance program. In the client terminal 12, the output control unit 96 performs control to output screen data generated by the screen data generation unit 92 to a display 28A and display the display screen 15 instead of distributing the screen data.

Thus, the medical assistance device of the invention can be implemented in various ways. In addition, the hardware configuration of a computer system, such as the medical assistance servers 11 and 126 and the client terminal 12, can be modified in various ways. For example, in order to improve the capacity or reliability, each of the medical assistance servers 11 and 126 may be configured by a plurality of server computers separated as hardware. Thus, the hardware configuration of a computer system can be appropriately changed according to the required performance, such as capacity, safety, or reliability. Needless to say, in order to ensure the safety or reliability, a basic program or a diagnostic assistance program may be duplicated or may be stored in a plurality of storage devices so as to be distributed, without being limited to hardware.

### Tenth Embodiment

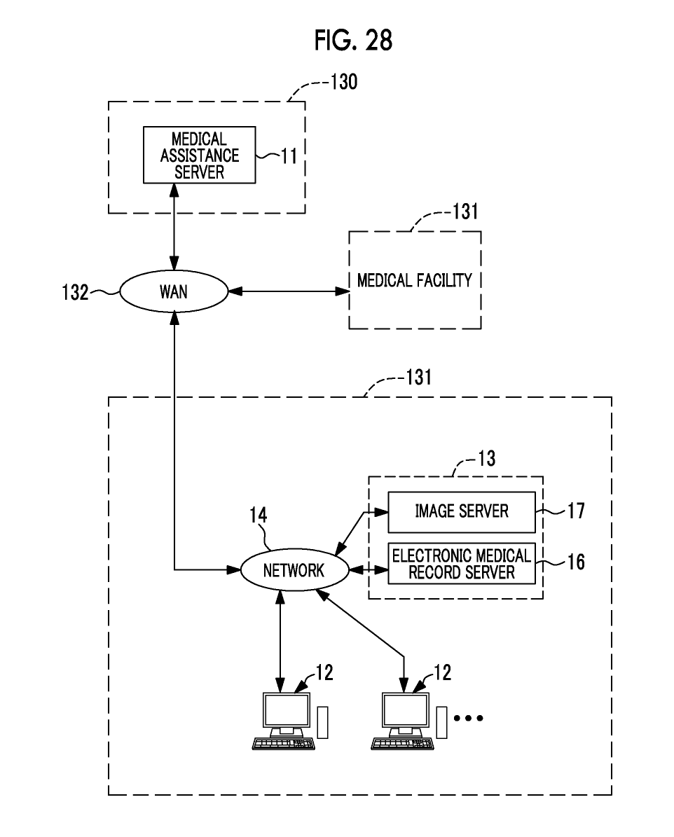

In each of the above embodiments, an example has been described in which the medical assistance servers 11 and 126 are used in one medical facility. However, as shown in FIG. 28, for example, one medical assistance server 11 may be installed in an external data center 130 so that a plurality of medical facilities 131 can use the medical assistance server 11 in the data center 130.

The medical assistance server 11 is communicably connected to the client terminal 12, which is installed in each of the plurality of medical facilities 131, through a wide area network (WAN) 132, such as the Internet or a public communication network, for example. The medical assistance server 11 receives requests from the client terminals 12 in the plurality of medical facilities 131, and distributes medical information or diagnostic assistance information to the each client terminal or provides application services for association setting to each client terminal.

The locations or management entity of the data center 130 and the medical assistance server 11 may be one of the plurality of medical facilities 131, or may be a separate service company from the medical facilities 131, for example. When using a WAN, such as the network 132, it is preferable to build a virtual private network (VPN) or to use a communications protocol with a high security level, such as hypertext transfer protocol secure (HTTPS), in consideration of information security.

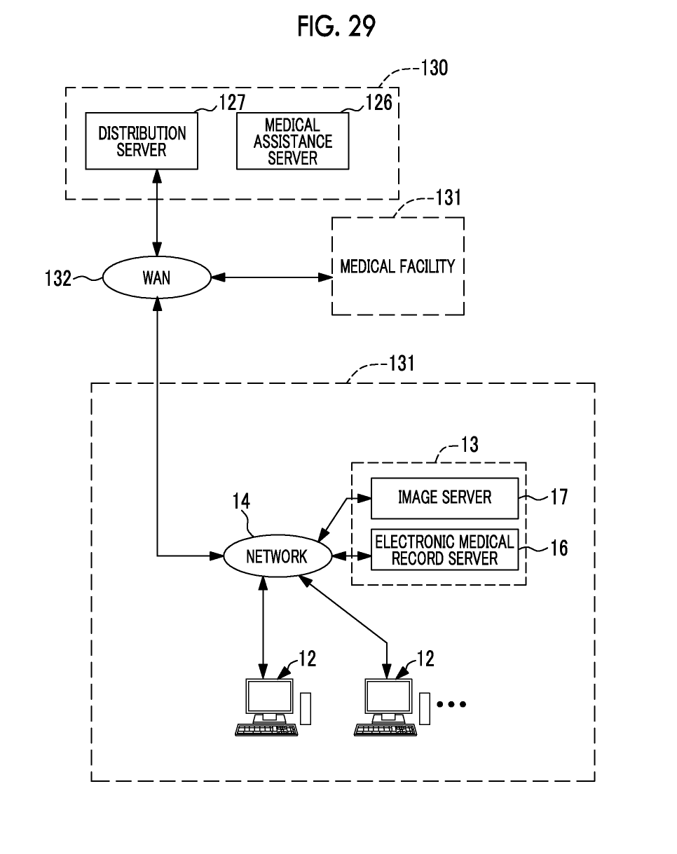

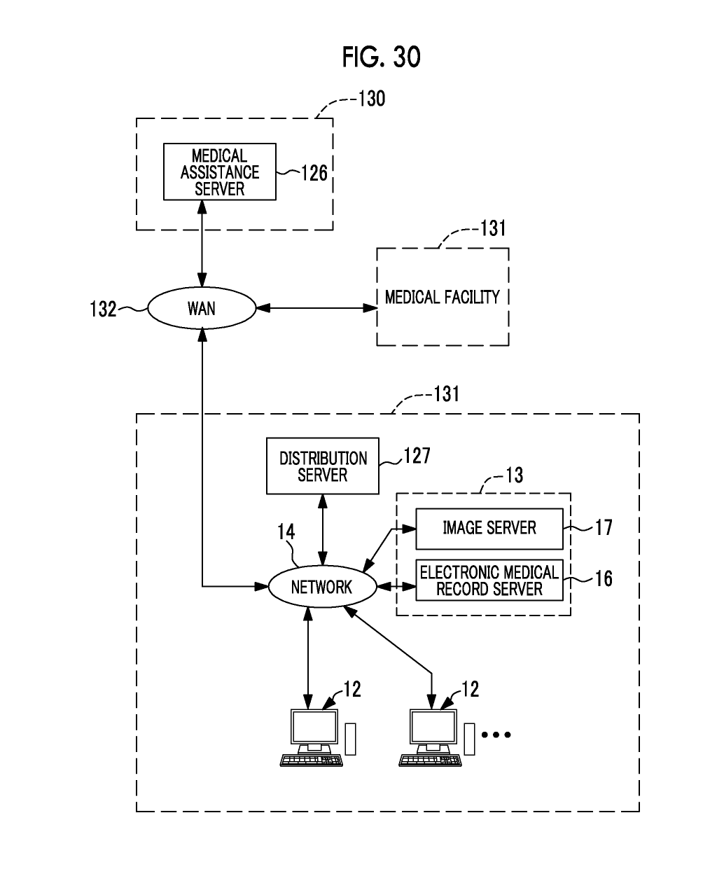

As shown in FIG. 29, instead of the medical assistance server 11, the medical assistance server 126 and the distribution server 127 shown in FIG. 26 may be provided in the data center 130. In addition, as shown in FIG. 30, the distribution server 127 may be provided in each medical facility 131, and the medical assistance server 126 may be provided in the data center 130 so that only the medical assistance server 126 is shared by the plurality of medical facilities 131.

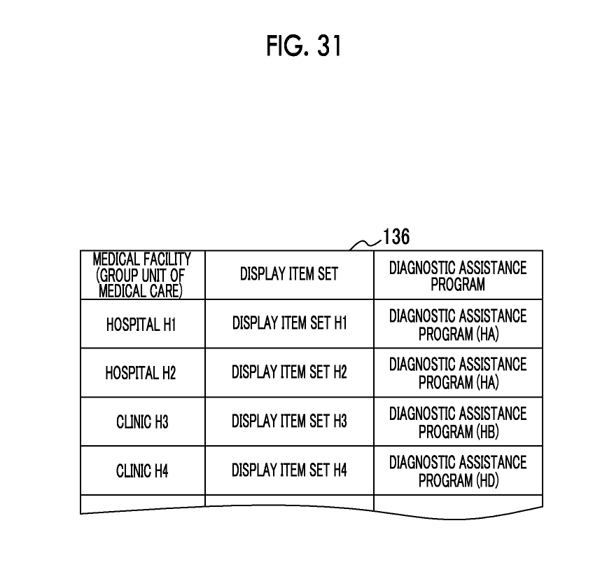

Thus, when providing the medical assistance servers 11 and 126 outside the medical facility 131, a medical facility may be set as a group unit of medical care as in a display item setting table 136 shown in FIG. 31. In the display item setting table 136, a display item set is set for each medical facility 130, such as hospitals H1 and H2 and clinics H3 and H4. Since medical equipment is different if the medical facilities 130 are different, display items that can be used in the medical facilities 130 are also different. The set display item set and a diagnostic assistance program are set so as to be associated with each medical facility 130.

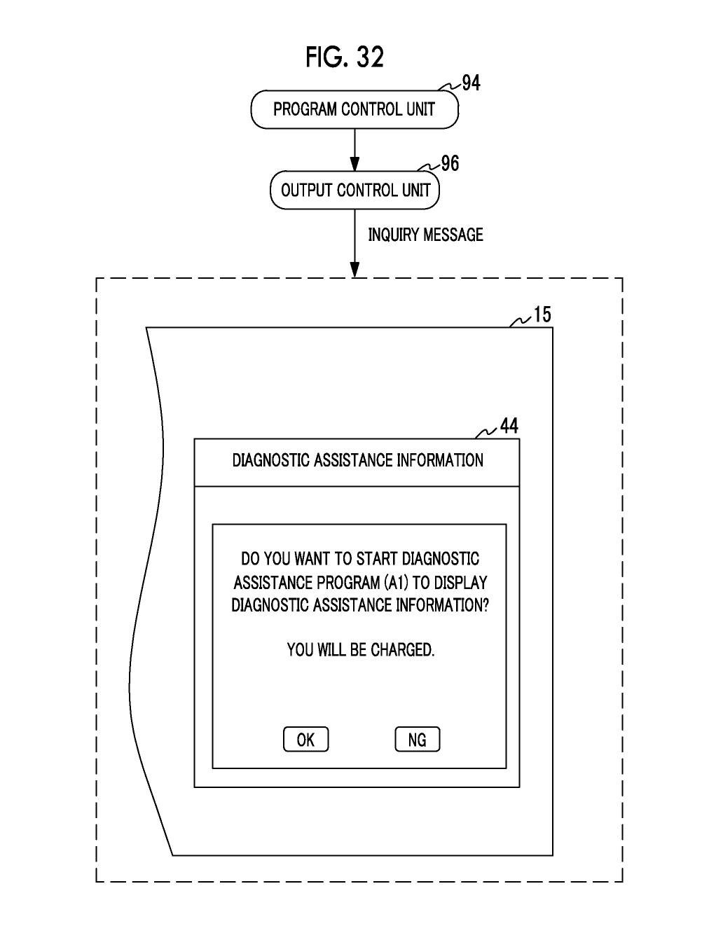

When the data center 130 in which the medical assistance servers 11 and 126 are installed is operated by the external service company, a usage-based system to charge for the use of a diagnostic assistance program according to the amount of use may be adopted. In this case, as shown in FIG. 32, for example, it is preferable to display a message asking whether or not to allow the starting of the diagnostic assistance program in the diagnostic assistance information display region 44 of the display screen 15 before starting the diagnostic assistance program. In addition to the inquiry message, it is preferable to display a message notifying that the user has to pay a charge for the service when the diagnostic assistance program is started. In this case, since a situation where charging is done while the user does not know is prevented, it is possible to perform proper charging. The program control unit 94 distributes such an inquiry message to the client terminal 12 through the output control unit 96.

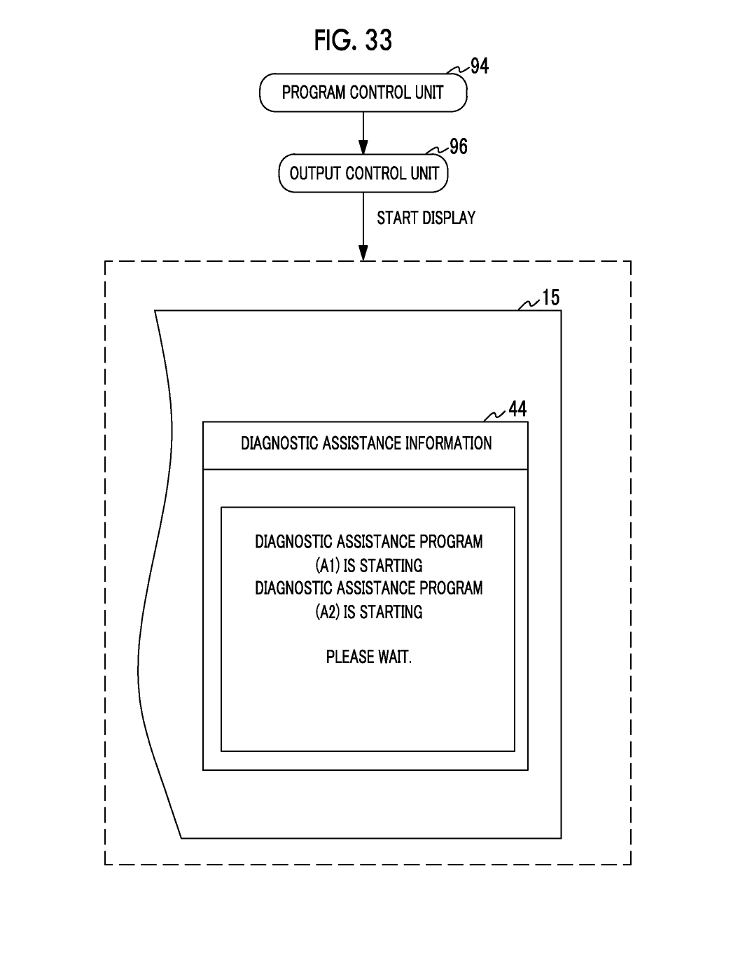

In addition, as shown in FIG. 33, when the diagnostic assistance program is starting, it is preferable to perform a start display, which indicates that the diagnostic assistance program is starting, in the display screen 15. In this case, the user can check the start display and stop the start so as not to be charged if not necessary.

Some medical facilities 130 may desire to receive the service of providing diagnostic assistance information only for a specific disease. In this case, it is possible to meet such a demand by using the start display shown in FIG. 33 or the inquiry message shown in FIG. 32.

In addition, as shown in FIG. 33, when a plurality of diagnostic assistance programs are starting, it is preferable to perform a start display so that it can be seen that there are a plurality of diagnostic assistance programs starting. In this case, it is possible to prevent a situation where the user checks only diagnostic assistance information displayed first and misses diagnostic assistance information displayed later.

It is needless to say that the invention is not limited to the above embodiments and various configurations can be adopted without departing from the scope of the invention. For example, it is also possible to appropriately combine the above-described various embodiments or various modifications. In addition to the program, the invention also extends to a storage medium for storing the program.

What is claimed is:

1. A medical assistance device, comprising:
an association setting unit that sets display items displayed on a display screen including medical information of a patient and a diagnostic assistance program for outputting diagnostic assistance information to assist diagnosis of the patient so as to be associated with each other for each group unit of medical care;
a medical information acquisition unit that acquires the medical information; and
a parameter output unit that outputs all pieces of first medical information corresponding to at least the display items in the medical information acquired by the medical information acquisition unit, as parameters to be input to the diagnostic assistance program, in a form in which all of the pieces of the first medical information are identifiable as the display items.

2. The medical assistance device according to claim 1,
wherein the group unit of medical care includes at least one of a medical facility, a department in a medical facility, a disease, a patient, and a medical practice phase indicating a progress phase of medical treatment or disease.

3. The medical assistance device according to claim 1,
wherein the first medical information and the diagnostic assistance information are displayed on the display screen.

4. The medical assistance device according to claim 3,
wherein the parameter output unit outputs the first medical information, and second medical information which does not correspond to the display items in the medical information, as parameters of the diagnostic assistance program.

5. The medical assistance device according to claim 4,
wherein the diagnostic assistance program has a function of outputting content regarding the first medical information and content regarding the second medical information as the diagnostic assistance information, and
the diagnostic assistance program has a function of outputting at least one of a message, which prompts a user to set at least one item of the second medical information to the display items, and evidence information as a basis of the message in case where the content regarding the second medical information is included in the diagnostic assistance information.

6. The medical assistance device according to claim 5,
wherein the medical information includes a plurality of types of examination images captured by a plurality of modalities, and
wherein the parameter output unit outputs the plurality of types of examination images as the first medical information or the second medical information.

7. The medical assistance device according to claim 6,
wherein, in case where there is image analysis information obtained by analyzing the examination images, the parameter output unit outputs the image analysis information together with the plurality of types of examination images.

8. The medical assistance device according to claim 3, further comprising:
a program control unit that controls starting of the diagnostic assistance program and the parameter output of the parameter output unit for the diagnostic assistance program to be started.

9. The medical assistance device according to claim 8, further comprising:
a designated information receiving unit that receives group unit of medical care designation information for designating the group unit of medical care, the group unit of medical care designation information being input through the display screen,
wherein the program control unit determines the diagnostic assistance program associated with the designated group unit of medical care based on the group unit of medical care designation information, and controls starting of the determined diagnostic assistance program and output of the parameters for the diagnostic assistance program to be started.

10. The medical assistance device according to claim 9,
wherein, in case where the group unit of medical care is a disease, the designated information receiving unit receives designation of multiple diseases, and
wherein in case where the multiple diseases are designated, the program control unit preferentially starts a diagnostic assistance program suitable for the multiple diseases among a plurality of the diagnostic assistance programs.

11. The medical assistance device according to claim 8,
wherein the program control unit starts the diagnostic assistance program, and executes a batch process for collectively outputting the diagnostic assistance information regarding a plurality of patients.

12. The medical assistance device according to claim 11,
wherein the diagnostic assistance program includes a diagnostic program having a function of outputting the diagnostic assistance information regarding one patient of interest, who is to be diagnosed, and reference information regarding other patients.

13. The medical assistance device according to claim 8,
wherein the program control unit periodically starts a monitoring program having a function of monitoring whether or not the first medical information is in a normal range, and performs a warning process in case where it is determined that the first medical information is not in the normal range.

14. The medical assistance device according to claim 3, further comprising:
an anonymization unit that anonymizes personal information of a patient in the first medical information.

15. The medical assistance device according to claim 3, further comprising:
a screen data generation unit that generates the display screen for displaying the first medical information based on the medical information acquired by the medical information acquisition unit; and
an output control unit that outputs the screen data.

16. The medical assistance device according to claim 15,
wherein a diagnostic assistance information display region for displaying the diagnostic assistance information is provided on the display screen.

17. The medical assistance device according to claim 15,
wherein a start display showing that the diagnostic assistance program is starting is performed on the display screen.

18. The medical assistance device according to claim 17,
wherein, when a plurality of the diagnostic assistance programs are starting, a start display showing that the plurality of diagnostic assistance programs are starting is performed.

19. The medical assistance device according to claim 15,
wherein a message asking whether or not to allow starting of the diagnostic assistance program is displayed on the display screen before starting the diagnostic assistance program.

20. An operation method of a medical assistance device, comprising:
an association setting step that sets display items displayed on a display screen including medical information of a patient and a diagnostic assistance program for outputting diagnostic assistance information to assist diagnosis of the patient so as to be associated with each other for each group unit of medical care;
a medical information acquisition step that acquires the medical information; and
a parameter output step that outputs all pieces of first medical information corresponding to at least the display items in the medical information acquired by the medical information acquisition unit, as parameters of the diagnostic assistance program, in a form in which all of the pieces of the first medical information are identifiable as the display items.

21. A non-transitory computer-readable recording medium on which a medical assistance program causing a computer to function as a medical assistance device is recorded, the program comprising:
an association setting step that sets display items displayed on a display screen including medical information of a patient and a diagnostic assistance program for outputting diagnostic assistance information to assist diagnosis of the patient so as to be associated with each other for each group unit of medical care;
a medical information acquisition step that acquires the medical information; and
a parameter output step that outputs all pieces of first medical information corresponding to at least the display items in the medical information acquired in the medical information acquisition step, as parameters of the diagnostic assistance program, in a form in which all of the pieces of the first medical information are identifiable as the display items.

22. A medical assistance system, comprising:
a diagnostic assistance program that outputs diagnostic assistance information to assist diagnosis of a patient; and
a medical assistance device that executes the diagnostic assistance program,
wherein the medical assistance device includes: an association setting unit that sets display items displayed on a display screen including medical information of a patient and the diagnostic assistance program so as to be associated with each other for each group unit of medical care;
a medical information acquisition unit that acquires the medical information; and
a parameter output unit that outputs all pieces of first medical information corresponding to at least the display items in the medical information acquired by the medical information acquisition unit, as parameters to be input to the diagnostic assistance program, in a form in which all of the pieces of the first medical information are identifiable as the display items.
